(19) United States
(12) Patent Application Publication
(10) Pub. No.: US 2004/0229785 A1
(43) Pub. Date: Nov. 18, 2004

(54) Screening methods to identify treatments for autoimmune disease

(76) Inventor: Denise Faustman, Weston, MA (US)

Correspondence Address: CLARK & ELBING LLP, 101 FEDERAL STREET, BOSTON, MA, 02110, US

(21) Appl. No.: 10/713,679

(22) Filed: Nov. 14, 2003

Related U.S. Application Data
(60) Provisional application No. 60/426,590, filed on Nov. 15, 2002.

(51) Int. Cl. G01N 33/567; A61K 38/17

(52) U.S. Cl. 514/12; 435/7.21

(57) ABSTRACT

The inventions features methods for screening test compounds for the potential to prevent, stabilize, or treat autoimmune diseases. The invention also features methods for diagnosing and monitoring autoimmune diseases.

### CROSS REFERENCE TO RELATED APPLICATIONS

This application claims benefit of U.S. Provisional Application No. 60/426,590, filed Nov. 15, 2002.

### BACKGROUND

The present invention relates to diagnostic methods for the detection and monitoring of autoimmune diseases. In autoimmune diseases, T cells are believed to act as causative agents that incorrectly recognize the host body as foreign. Diagnostic methods currently in use or proposed for the diagnosis of autoimmune disease are based on the detection or recognition of particular elements of an autoimmune response. Some examples include those in U.S. Pat. No. 6,638,771 which describes the detection of soluble liver antigen (SLA) auto-antibodies for the diagnosis of auto-immune hepatitis; U.S. Pat. No. 6,566,082 which describes the detection of T-cell co-stimulatory molecule OX-40; U.S. Pat. No. 6,545,137 which describes the detection of LDL-receptor related protein-3 for the diagnosis of Type I diabetes; U.S. Pat. No. 5,700,641 which describes the detection of anti-DNA antibodies in serum for the diagnosis of Lupus Erythematosus, Rheumatoid Arthritis, and Scleroderma; and U.S. Pat. No. 5,445,940 which describes monoclonal antibodies reactive with an epitope of the T cell receptor alpha chain variable region, Vα12.1, on human T lymphocytes for the diagnosis of rheumatoid arthritis. These methods are based on specific static characteristics (e.g., autoantibodies, cell surface markers, or oligonucleotides) of the molecules or cells that characterize an autoimmune response, with many of them specific for the diagnosis of one particular autoimmune disease or a limited set of closely related autoimmune diseases. What is needed is a general method for the detection of autoimmune disease, or a subject's risk of developing such a disease, based on autoimmune cell behavior. Optimally, this method could also be used to identify compounds that can be used to prevent, stabilize, or treat autoimmune disease.

### SUMMARY OF THE INVENTION

Accordingly, in a first aspect the invention features a method for screening a test compound for the potential to prevent, stabilize, or treat an autoimmune disease. The method includes the steps of contacting a blood sample from a mammal having, or at risk for developing, an autoimmune disease with the test compound and measuring the viability of leukocytes in the sample. The compound is determined to have potential therapeutic efficacy if the viability of the leukocytes in the blood sample decreases relative to the viability of leukocytes in a control blood sample that has also been contacted with the compound. The control sample is from a mammal of the same species not having or not being predisposed to the autoimmune disease.

In one embodiment, the leukocytes overexpress a receptor for a chemokine from the group that includes FasL, TNF-alpha, IL-1beta, IL-6, IL-12, and IFN-gamma (γ-interferon). In another embodiment, the leukocytes are deficient in the expression of CD 180 (RP 105). By deficient is meant that the receptor or ligand is expressed on the cell surface at levels that are less than 50%, 40%, 30%, 20%, 10%, or 5% that which would be normally be expected, or that the leukocyte does not express any detectable amounts of the receptor/ligand (e.g., by FACS analysis). In yet another embodiment, the leukocytes include autoimmune cells, such as, for example, those splenocytes, T lymphocytes, B lymphocytes, or cells of bone marrow origin that participate in an autoimmune response.

In addition to cell population counts, leukocyte viability can also be measured by apoptotic or necrotic events. Apoptotic or necrotic events are known by those skilled in the art to confer certain characteristics to those cells which are undergoing a process of apoptosis or necrosis. Methods for screening test compounds can also include the use of inhibitors of apoptosis or necrosis. An example of their use as applied to the first aspect includes the steps of: i) partitioning a blood sample of a mammal having, or at risk for developing, an autoimmune disease into a first fraction which is contacted with an inhibitor of apoptosis or necrosis before contacting it with the test compound and a second fraction which is not contacted with the inhibitor of apoptosis/necrosis before contacting it with the test compound; ii) after both first and second fractions are contacted with the test compound, measuring the ratio of leukocyte viability in the first fraction of step i) to the leukocyte viability of the second fraction of step i); iii) partitioning a second blood sample from a mammal not having or not being predisposed to the autoimmune disease into a third fraction which is contacted with an inhibitor of apoptosis or necrosis before contacting it with the test compound and a fourth fraction which is not contacted with the inhibitor of apoptosis/necrosis before contacting it with the test compound; and iv) after both third and fourth fractions are contacted with the test compound, measuring the ratio of leukocyte viability in said third fraction of step iii) to the leukocyte viability in said fourth fraction of step iii). Since the inhibition of apoptosis/necrosis pathways affords greater protection to autoimmune-related leukocytes relative to non-autoimmune related leukocytes, a compound is determined to have potential therapeutic efficacy if said ratio of step ii) is greater than the ratio of step iv).

In another aspect, the invention features a method for screening a test compound for the potential to have therapeutic efficacy for preventing, stabilizing, or treating an autoimmune disease that includes the steps of contacting a blood sample from a mammal having, or at risk for developing, an autoimmune disease with the test compound followed by measuring the viability of autoimmune cells relative to a blood element in the sample. The compound is determined to have potential therapeutic efficacy if the viability of autoimmune cells to which it has been contacted is decreased relative to the blood element.

In an embodiment, the population of autoimmune cells is partitioned into a first fraction which is contacted with the test compound and a second fraction which is not contacted with the test compound. The ratios of viable autoimmune cells of the first and second fractions to the second blood element are measured and the compound is determined to have potential therapeutic efficacy if the ratio of autoimmune cells contacted with the compound is less than the ratio of autoimmune cells not contacted with the compound.

By “blood element” is meant any substance that can be measured in blood. A blood element can be naturally occurring or introduced as a part of an assay. Some exemplary blood elements are, for example, white blood cells, red blood cells, minerals, such as sodium, potassium, or calcium, hormones, sugars, amino acids, steroids, such as cholesterol, and vitamins. When the blood element that is being measured as a control is a blood cell, total cell count, or a count that is reflective of a subset of cells, can be used for comparison purposes.

In another aspect, the invention features a method for diagnosing an autoimmune disease or a predisposition to an autoimmune disease in a mammal, desirably a human, that includes the steps of contacting a blood sample from the mammal with a compound that preferentially decreases the viability of leukocytes and then measuring leukocyte cell viability. The mammal is determined to have an autoimmune disease, or a predisposition for an autoimmune disease, if the viability of the leukocytes in the blood sample which has been contacted with the compound is decreased relative to the viability of leukocytes in a control blood sample that has also been contacted with the compound. The control sample is from a mammal of the same species not having or not being predisposed to the autoimmune disease.

In another aspect, the invention features a method for diagnosing an autoimmune disease or a predisposition to an autoimmune disease in a mammal, desirably a human, that includes the steps of contacting autoimmune cells from the mammal with a compound that preferentially decreases the viability of autoimmune cells and measuring autoimmune cell viability relative to a second blood element from the mammal which has also been contacted with the compound in a similar manner. The mammal is determined to have an autoimmune disease, or a predisposition for an autoimmune disease, if the viability of the autoimmune cells that have been contacted with the compound is decreased relative to the blood element. In one embodiment, the blood element can be a non-autoimmune leukocyte. In another embodiment, the blood element can be an erythrocyte.

One example includes the following steps: obtaining a control blood sample from the mammal before administration of the compound; measuring the ratio of viable autoimmune cells to a second blood element in the control sample; administering a compound that preferentially decreases the viability of autoimmune cells to the mammal; obtaining another sample from the mammal; and measuring the ratio of viable autoimmune cells to the second blood element in the second sample. A decrease in the ratio of viable autoimmune cells to the second blood element in the second blood sample relative to the same ratio obtained in the control blood sample is indicative of the mammal having an autoimmune disease or being predisposed to the autoimmune disease.

For any of the methods of the present invention which employ the use of a compound that preferentially decreases the viability of autoimmune cells, the compound can be TNF-alpha, an agonist for the TNF-alpha receptor, such as, for example, a human or humanized monoclonal antibody agonist, or a compound that can selectively decreases the viability of autoimmune cells by inducing TNF-alpha. Such inducing compounds include, for example, complete Freund's adjuvant (CFA), ISS-ODN, microbial cell wall components with LPS-like activity, cholera particles, E. coli heat labile enterotoxin, E. coli heat labile enterotoxin complexed with lecithin vesicles, ISCOMS-immune stimulating complexes, polyethylene glycol, poly(N-2-(hydroxypropyl)methacrylamide), synthetic oligonucleotides containing CpG or CpA motifs, monophosphoryl lipid A, Bacillus Clamette-Guerin, γ-interferon, Tissue Plasminogen Activator, LPS, Interleukin-1, Interleukin-2, UV light, a lymphotoxin, cachectin, a TNFR-2 agonist, an intracellular mediator of the TNF-alpha signaling pathway, a NFκB inducing substance, IRF-1, STAT1, a lymphokine, or the combination of TNF-alpha and an anti-TNFR-1 antibody. Preferably, TNF-alpha, a human or humanized monoclonal antibody TNF-alpha receptor agonist, CFA, gamma-interferon, or BCG is used.

Compounds that can also decrease the viability of autoimmune cells are those that bind to Toll, Toll-like receptors (TLRs), MD-1, or Ly78. These compounds include small molecule or antibody agonists of TLR1 (such as, for example, triacetylated lipopeptides (LP), phenol-soluble modulin, or OspA LP from B. burgdorferi), small molecule or antibody agonists of TLR2 (such as, for example, LP with TLR1 or TLR6, or HSP60 with TL4), small molecule or antibody agonists of TLR3 (such as, for example, double-stranded RNA), small molecule or antibody agonists of TLR4 (such as, for example, LPS from Gram-negative bacteria, HSP60, mannuronic acid polymers, flavolipins, tecihuronic acids, neumolysin, fimbriae, surfactant protein A, hyaluronan, oligosaccharides, heparin sulfate fragments, fibrinogen peptides, or beta-defensin-2), small molecule or antibody agonists of TLR5 (such as, for example, flagellin), small molecule or antibody agonists of TLR6 (such as, for example, deacetylated LP or phenol-soluble modulin), small molecule or antibody agonists of TLR7 (such as, for example, imidazolquinoline anti-virals), small molecule or antibody agonists of TL8 (such as, for example, imidazolquinoline) or small molecule or antibody agonists of TLR9 (such as, for example, bacterial DNA as CpG motifs). In one example of a compound used to decrease the viability of autoimmune cells via a TLR, BCG is used to target a subpopulation of B cells that are CD180-deficient.

Another aspect of the invention features a method for the stratification of a human patient into a therapeutic subgroup for an autoimmune disease. The method includes the following steps: contacting a blood sample from the patient with a compound that preferentially decreases the viability of leukocytes; measuring the cell viability of the leukocytes; and placing the patient into a therapeutic subgroup based on the amount of decrease of leukocyte viability.

In one embodiment, leukocyte cell viability is measured relative to the cell viability of leukocytes in a control blood sample obtained from a human that does not have, or is not at risk for developing, the autoimmune disease. The control blood sample and the blood sample from the patient are contacted with the leukocyte-killing compound in the same manner and therapeutic subgroup placement is based on the relative amount of decrease of leukocyte viability. In another embodiment, leukocyte viability is determined relative to a second blood element in the sample. Both the blood sample and the second blood element from the patient are contacted with the leukocyte-killing compound in the same manner and therapeutic subgroup placement is based on the relative amount of decrease of leukocyte viability.

In another embodiment, the compound is administered to the patient. One example includes the following steps: obtaining a control sample from the patient before administration of the compound; measuring the ratio of viable autoimmune cells to a blood element in the control sample; administering a compound that decreases the viability of autoimmune cells to the patient; obtaining another sample from the patient (e.g., within 24 hours after administering the compound, preferably within 6-12 hours after administering the compound); and measuring the ratio of viable autoimmune cells to the blood element in the second sample, wherein an decrease in the ratio of viable autoimmune cells to the blood element in the second sample relative to the same ratio obtained in the control sample determines the severity or course of the disease.

In a related aspect, the invention features a method for monitoring a therapy for a human patient with an autoimmune disease or a predisposition to an autoimmune disease. The method includes steps of contacting a blood sample from the patient with a compound that preferentially decreases the viability of leukocytes and measuring the cell viability of the leukocytes. One example includes the following steps: i) obtaining a first blood sample from the patient and contacting it with a compound that preferentially decreases the viability of leukocytes; ii) measuring the viability of leukocytes in the first blood sample; iii) after a period of time (e.g., 12 hours, 1 day, 2 days, 1 week, 2 weeks, 1 month), obtaining a second blood sample from the patient and contacting the second blood sample with the compound; iv) measuring the viability of leukocytes in the second blood sample; and v) determining the efficacy of the therapy based on leukocyte viability, where an increase in leukocyte viability over time indicates that the therapy is efficacious. The monitoring method just described can further include obtaining additional blood samples after obtaining the second blood sample at appropriate intervals of time, as described above, over the course of the therapy.

In one embodiment, leukocyte cell viability is measured relative to the cell viability of leukocytes in a control blood sample obtained from a human that does not have, or is not at risk for developing, the autoimmune disease. The control blood sample and the blood sample from the patient are contacted with the leukocyte-killing compound in the same manner and therapeutic subgroup placement is based on the relative amount of decrease of leukocyte viability. In another embodiment, leukocyte viability is determined relative to a second blood element in the sample. As before, both the blood sample and the second blood element from the patient are contacted with the leukocyte-killing compound in the same manner. In yet another embodiment, the compound is administered to the patient.

For any of the diagnostic methods of the invention, the autoimmune disease can be Alopecia, Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, Wegener's Granulomatosis, or myasthenia gravis.

Definitions

What is meant by “apoptotic event” is an event that is characteristic of programmed cell death (apoptosis). Apoptosis is an active process signaled by the nucleus of a cell and can be characterized by cell shrinkage or by the loss of integrity of the cell membrane. Apoptosis can also be characterized by the cleavage of DNA into fragments or by the degree of expression of intracellular enzymes, such as caspases, known to contribute to programmed cell death.

By “autoimmune disease” is meant a disease in which an immune system response is generated against self epitopes. Some examples of autoimmune diseases include Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiff-Man Syndrome, Takayasu Arteritis, Temporal Arteritis/Giant Cell Arteritis, Ulcerative Colitis, Uveitis, Vasculitis, Vitiligo, Wegener's Granulomatosis, and myasthenia gravis.

Desirable autoimmune cells are defective in protection from apoptosis and/or necrosis. This defect in protection from apoptosis can be in the pathway linked to TNF-induced apoptosis, or an apoptotic pathway unrelated to TNF. Autoimmune cells of the present invention include, for example, adult splenocytes, T-lymphocytes, B-lymphocytes, and cells of bone marrow origin, such as defective antigen presenting cells of a mammal. In various embodiments, cells capable of expressing MHC class I and peptide are cells that are class I+/+ or cells that are class I^{-/-} (e.g., cells having a mutation in the β2M gene) but that are reconstituted in vivo by a compensatory component (e.g., serum β2M).

By “blood sample” is meant a sample containing blood cells. A blood sample can include whole blood or blood fractions, such as leukocytes isolated from whole blood. A blood sample can be peripheral blood in circulation in a mammal or a blood phlebotomized from a mammal.

What is meant by “cell viability” is the ability for a cell to function normally. The viability of a cell can be decreased by the initiation of an apoptotic or a necrotic event. Cell viability can be measured by the biochemical characterization of cells undergoing apoptotic and/or necrotic processes, for example, by the cleavage of DNA into fragments or by the degree of expression of intracellular enzymes known to participate in necrosis or apoptosis. Cell viability can also be measured by events that occur on the cell surface membrane such as, for example, the disordering of the membrane. Cell viability can also be measured by the examination of cell population, wherein a decrease in the cell population of a particular cell type relative to a control is indicative of prior apoptotic or necrotic events resulting in cell death.

By “leukocyte” is meant a white blood cell. Leukocytes can be neutrophiles, macrophages, or lymphocytes, such as T-cells, B-cells, or other blood cells of the immune system.

Leukocytes obtained from a subject not having and not at risk for developing an autoimmune disease are those obtained within a one month period from a subject who has undergone diagnostic testing specific for the autoimmune disease, with the test results indicating an absence of disease or a lack of predisposition for the disease in the subject.

“MHC class I and peptide” is commonly understood to refer to the MHC/peptide complex as it is naturally presented on the surface of a cell in connection with the normal functioning of the immune system. Cytoplasmic antigens are processed into peptides by cytoplasmic peptides by proteases and the proteasome, a multicatalytic proteinase complex associated with the Lmp2, Lmp7, and Lmp10 protein. MHC class I multimers are also known and have been used to provide an approach to the visualization of antigen-specific T-cells, including autoimmune cells. For example, MHC class I multimers labeled with a fluorescent tag can be useful in the flow cytometric analyses of autoimmune cells, as shown by Sun, et al. in Arthritis Res. 3:265-9 (2001) and Bill, et al. in Arthritis Res. 4:261-5 (2002)

Receptor expression can be determined by the number of cell surface or intracellular receptors or by a measurement of receptor activity, with overexpression characterized by a greater number or increased activity of the receptor relative to that found in an unstimulated cell.

By “selectively killing blood cells” is meant directly or indirectly reducing the number or relative percentage of a subpopulation of susceptible blood cells (e.g., T-cells). Desirably, the subpopulation is a subset of T-cells, B-cells, or macrophages, with susceptible memory T-cells being autoimmune T-cells, i.e., T-cells that are activated by presented self epitopes. In desirable embodiments, the susceptible naïve T-cells are cells that would otherwise become autoimmune T-cells. Desirably, the number of autoimmune T-cells or cells that would otherwise become autoimmune T-cells decreases by at least 25, 50, 100, 200, or 500% more than the number of non-susceptible cells which are used as a control cell population. In some embodiments, the number of autoimmune T-cells or cells that would otherwise become autoimmune T-cells decreases by at least 25, 50, 75, 80, 90, 95, or 100%, as measured using standard methods. The T-cells can be killed due to any pathway, such as apoptosis, necrosis, and/or activation induced cell death. Apoptosis can be assayed by detecting caspase-dependent cell shrinkage, condensation of nuclei, or intranuclear degradation of DNA. Necrosis can be recognized by caspase-independent cell swelling, cellular degradation, or release of cytoplasmic material. Necrosis results in late mitochondrial damage but not cytochrome C release. In some embodiments, memory T-cells are killed by apoptosis and naive T-cells are killed by necrosis. The decrease in a population of susceptible cells in a sample treated with a compound that induces cell death by one of the pathways indicated above can be measured by accounting for the decrease in cells relative to a population of cells that unaffected, or affected to a lesser extent by the compound. One method of quantitating susceptible cells is by using cell markers or biological events particular to those cells. Such markers can be intracellular or expressed on the cell surface membrane.

By “stimulated blood cell” is meant a blood cell (e.g., a memory T-cell, a B-cell, or a macrophage) that has been exposed to an antigen, including “self” antigens that generate an autoimmune response.

By “compound” is meant one compound or a mixture of compounds. The identity of a compound can be known or unknown. If “compound” refers to a mixture of compounds, this mixture can be from a biological sample. “Compound” can also represent a combinatorial library of compounds, including a phage display library.

By “unstimulated blood cell” is meant a blood cell (e.g., a naïve T-cell, a B-cell, or a macrophage) that has not been exposed to an antigen.

Stimulated cells tend to be in later stages of maturation than unstimulated cells, in active progression through the cell-cycle, and/or involved in infiltrating a diseased or damaged organ or tissue. Unstimulated cells tend to progress through the cell-cycle more slowly or not at all. Memory T-cells tend to express a higher density of IL-2 receptor (e.g., 10-20% higher density) than naïve T-cells. Naïve T-cells tend to express a higher density (e.g., a 5-20% higher density) of CD45RB^{kow}, CD45RB^{high}, CD95, and/or CD62L than memory T-cells.

Other features and advantages of the invention will be apparent from the following description of the preferred embodiment thereof, and from the claims.

### BRIEF DESCRIPTION OF THE DRAWINGS

FIG. 1 is a list of exemplary death receptors.

FIG. 2. is a list of exemplary inhibitors of cell death.

FIG. 3 is a list of standard kits that can be used to measure the level of cell death.

FIG. 4 is list of exemplary compounds that induce cell death.

FIG. 5 is a graph in which cell death is quantified over time for splenocytes from C57BL/6 (B6) and pre-diabetic NOD mice and kept in tissue culture at 37° C.

FIG. 6 is a graph comparing the cell viability vs. cell density for both NOD and B6 splenocytes which had been purified by removing RBCs with a via a magnetic affinity purification technique (Terl 19) and cultured for 24 hours.

FIG. 7 is a graph comparing splenocyte-viability for the three methods of RBC removal used in the methods of the invention (NH₄Cl lysis, ficoll-paque gradient, and Terl 19 MACS beads).

FIGS. 8A-8D are graphs resulting from flow cytometric experiments on splenocytes. FIG. 8A shows Ter119-purified B6 splenocytes stained with Annexin V and PI, and analyzed on by Forward Scatter flow cytometry (FSC) and Side Scatter flow cytometry (SSC). FIG. 8B is an ungated Annexin V versus PI plot showing a typical distribution of cells that remained unstained (live) and cells that stained with both Annexin V and PI (late apoptotic/dead). FIGS. 8C and 8D are gated studies, plotting the Annexin V/PI regions R1 and R3, respectively.

FIG. 9A is a graph that shows cell viability at various cell densities for separate populations of T-cells (purified by using a PanT magnetic column) and non-T-cells (purified by using a CD90 magnetic column) obtained from both C57B1/6 and NOD mice.

FIG. 9B are flow cytometry plots showing the viability of splenocytes from a C57BL/6 mouse purified in three different ways to leave a mix of both T cells and non-T cells (purified by using only a Ter-119 column and labeled “T 11”); only T cells (purified by using a PanT magnetic column and labeled “Pan”); or only Non-T cells (purified using CD90+ Ter-119 columns and labeled “CD90/Ter11”).

FIG. 10 is a graph that shows the difference in sensitivity between B6 and NOD splenocytes to TNF-α as analyzed by flow cytometry (the ordinate axis represents percent viability of the cells).

FIG. 11 is an analysis of NOD vs. B6 T cells (purified by using a PanT age of the patient.

FIGS. 15A and 15B are flow cytometry plots showing a significant increase in apoptosis of cells contacted with TNF-α in vitro for CD3, CD19, and CD11b positive PBLs isolated from two human subjects with Type I diabetes.

FIG. 16 is a graph showing the sensitivity of T cell populations from NOD mice as analyzed by an automated assay method of the invention.

FIGS. 17A-17C are comparisons of cell viability with histology results for an NOD mouse that is symptomatic of disease (FIG. 17A), an NOD mouse the is not symptomatic of disease (FIG. 17B), and a control B6 mouse (FIG. 17C).

FIG. 18A is a graph showing the viability of non-T cells using an automated assay method of the invention.

FIG. 18B is a graph comparing the change in viability of non-T cells to TNF-α treatment.

### DETAILED DESCRIPTION

Role of Cell Death in Autoimmune Disease

A unique characteristic of autoimmune cells is that they frequently exhibit heightened apoptosis sensitivity. This sensitivity in the memory cell can be potentiated by TNF-α due to a link to NF-KB and proteasome defects. According to the present non-limiting theory of the invention, multiple cell death pathways exist in a cell, and any one or more of these cell death-related pathways are defective in autoimmune cells, accentuating the sensitivity of these cells to cell death. For example, susceptibility to TNF-alpha-induced apoptosis could occur via a failed cell death inhibition pathway (e.g., by defective production and activation of the transcription factor NF-κB, as in the NOD mouse). Furthermore, it is known that there are two different TNF-alpha

### DETAILED DESCRIPTION

Role of Cell Death in Autoimmune Disease

A unique characteristic of autoimmune cells is that they frequently exhibit heightened apoptosis sensitivity. This sensitivity in the memory cell can be potentiated by TNF-α due to a link to NF-κB and proteasome defects. According to the present non-limiting theory of the invention, multiple cell death pathways exist in a cell, and any one or more of these cell death-related pathways are defective in autoimmune cells, accentuating the sensitivity of these cells to cell death. For example, susceptibility to TNF-alpha-induced apoptosis could occur via a failed cell death inhibition pathway (e.g., by defective production and activation of the transcription factor NF-κB, as in the NOD mouse). Furthermore, it is known that there are two different TNF-alpha receptors. Defective signaling through either receptor could render autoimmune cells susceptible to TNF-alpha induced apoptosis. In other examples, defective cell signaling through surface receptors that stimulate pathways that interact with the cell death pathway, e.g., LPS, IL-1, TPA, UV light etc., could render autoimmune cells susceptible to cell death.

Diabetes as manifested in a NOD mouse shares numerous features with human Type I (insulin-dependent) diabetes. In both syndromes, faulty antigen presentation occurs via the MHC Class I pathway and there is an excess of T-cells displaying markers of developmental immaturity, e.g., CD ₄₅RB^{high}, CD62L, and CD95. In an NOD mouse that has received successful disease therapy, these abnormalities revert toward normal. One feature of the NOD mouse is the occurrence of enhanced susceptibility to TNF-α-induced apoptosis in certain lymphoid lineages. This feature, which is readily evident after 10 weeks of age in splenocytes of NOD mice that progress to hyperglycemia, can also be abolished by successful therapy.

Data from the NOD mouse model of autoimmune diabetes and data from peripheral blood lymphocytes (PBL) of patients with Type I diabetes support a shared immunologic abnormality, specifically an increased susceptibility to apoptosis during in vitro incubation with TNF-α. In the NOD model, the cells that are responsible for the autoimmune destruction of pancreatic islets are TNF-α sensitive. It is worth noting that differences between the in vitro behavior of NOD splenocytes and isolated human PBL include: (i) spontaneous hPBL death during 24 hour culture compared with relatively stable viability of NOD splenocytes and (ii) an unpredictable time course of TNF-α induced apoptosis in human PBLs compared with NOD splenocytes. The variability of the results obtained, the inability to perform repeated assays on the same sample, the inability to perform kinetic studies of the time course of TNF-α sensitivity, and the relatively long time necessary for sample preparation and analysis currently hamper the use of flow cytometry for monitoring the “real-time” response to human autoimmune treatment therapies. Even with these limitations, studies of human PBLs from non-diabetic controls and Type I diabetic patients have revealed increased apoptosis after in vitro incubation of blood samples with TNF-α in several cell lineages from type I diabetic patients, with at least 45% of a randomly selected group of type I diabetic patients having a heightened level of apoptosis of PBLs after TNF-α incubation.

Described herein are simplified methods for isolating human PBLs and high-throughput screening methods that can be used in epidemiologic and interventional studies. Specifically, these methods can (i) decrease the time required to isolate PBLs for subsequent analysis compared to traditional methods that typically require over 10 hours and may result in cell loss prior to contacting the sample with compound; (ii) examine specific cell lineages and determine the most efficient markers of programmed cell death for in vitro assays; (iii) measure the effectiveness of inducers of cell death and cell cycle regulators; (iv) investigate the kinetics and dosing of inducers of cell death to determine the most useful, precise time points and doses for application in human studies; and (v) enable a large number of assays to be preformed reliably with a minimum of cell manipulation.

Screening and Identifying Compounds for the Treatment of Autoimmune Disease

Accordingly, one aspect of the present invention features methods for identifying compounds that induce cell death of autoimmune cells and are thus useful for the treatment, prevention, or stabilization of autoimmune disease. These methods involve contacting a candidate compound with a blood sample (e.g., a whole blood sample or leukocytes isolated from a blood sample that are then, desirably, placed in an alginate solution or matrix) from a mammal having, or at risk for developing, an autoimmune disease and determining whether treated cells are less viable than a control substance that has been treated with the same compound. Examples of control substances include leukocytes from a non-autoimmune-diseased mammal of the same species or a second blood element. The second blood element is not susceptible to cell death via an aberrant apoptosis or necrosis pathway inherent to autoimmune cells and can be obtained from the autoimmune-diseased mammal. Desirable examples include non-autoimmune leukocytes or erythrocytes. A test compound has therapeutic potential for autoimmune disease therapy when the viability of leukocytes is decreased relative to the measurement of the control substance.

The targeted leukocytes can include autoimmune cells. Desirably, the autoimmune cells are splenocytes, T lymphocytes, B lymphocytes, or cells of bone marrow origin. A description of autoimmune effectors, receptors, and factors can be found in Textbook of Autoimmune Diseases, edited by Lahita, et al., Lippincott Williams & Wilkins, 1st edition (2000). For any of the screening or diagnostic methods of the present invention, the targeted leukocytes can overexpress a receptor for a chemokine from the group consisting of: FasL, TNF-alpha, IL-1beta, IL-6, IL-12, and IFN-gamma. Potentially susceptible autoimmune leukocytes can overexpress a number of receptors or factors, some of which are described in Roitt's Essential Immunology, Roitt, et al., Blackwell Science Inc., 10th edition (2001). Potentially susceptible leukocytes can also be characterized by the underexpression of certain receptors or factors. For example, if the targeted leukocyte is a B-cell, it may be deficient in the expression of CD180 (RP105).

The ability of a compound to promote cell death of autoimmune cells, but desirably not promote cell death of normal cells, can also be measured by determining the effect of different concentrations of the compound on cells from a mammal's blood. If the compound specifically compromises leukocytes that have an increased susceptibility to cell death, the number of cells that are compromised due to exposure to the compound initially increases as a function of the amount of compound that is added in a typical dose-response fashion. At higher doses, the number of cells that die plateaus because the most susceptible cells have died and the living cells are desirably resistant to the compound. If the amount of compound that is administered is further increased, more cells may compromised due to effect of the compound on the remaining cells (e.g., non-autoimmune cells that are resistant to lower levels of the compound but that may be compromised in the presence of higher levels of the compound).

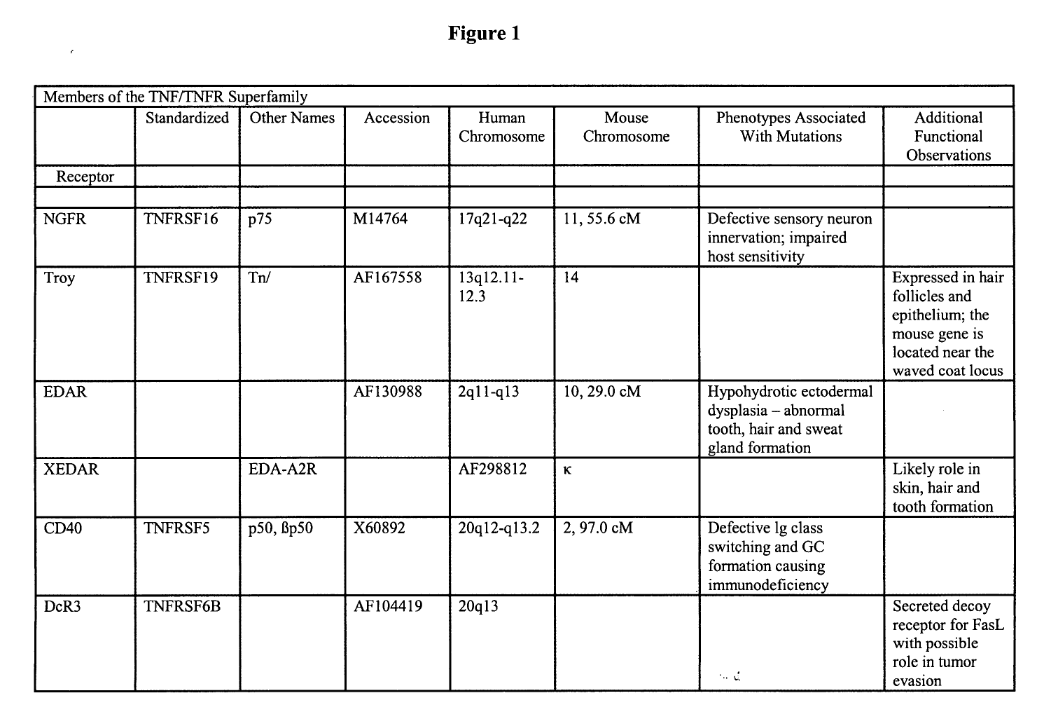

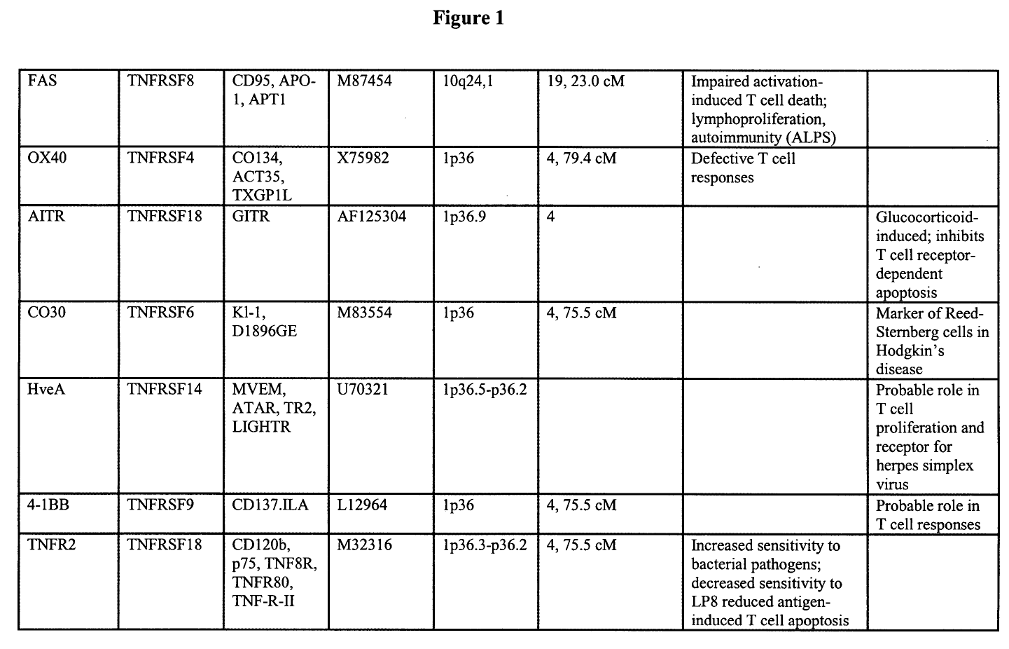

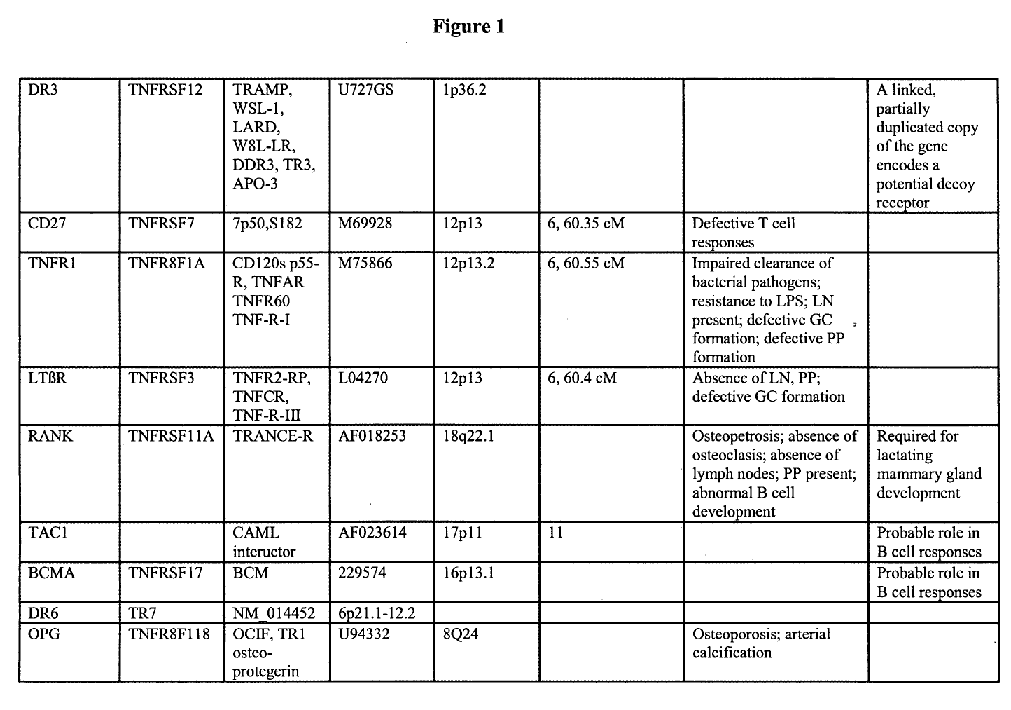

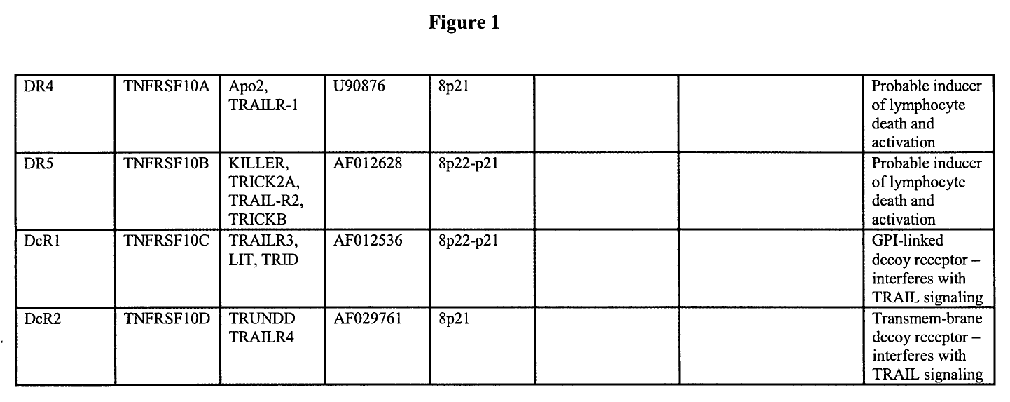

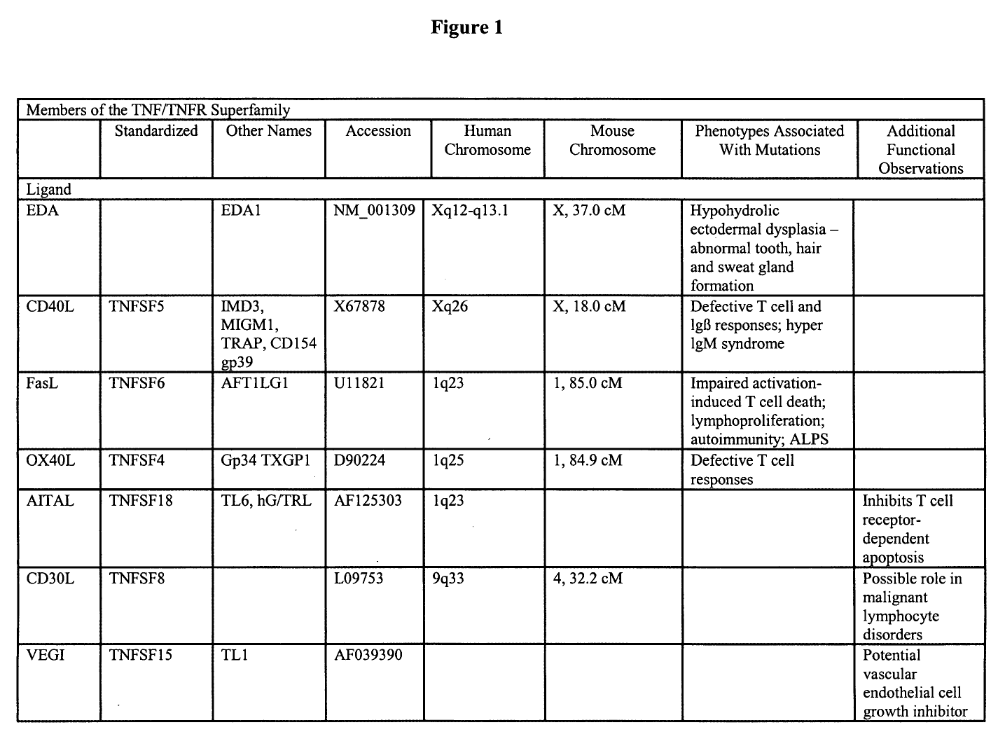

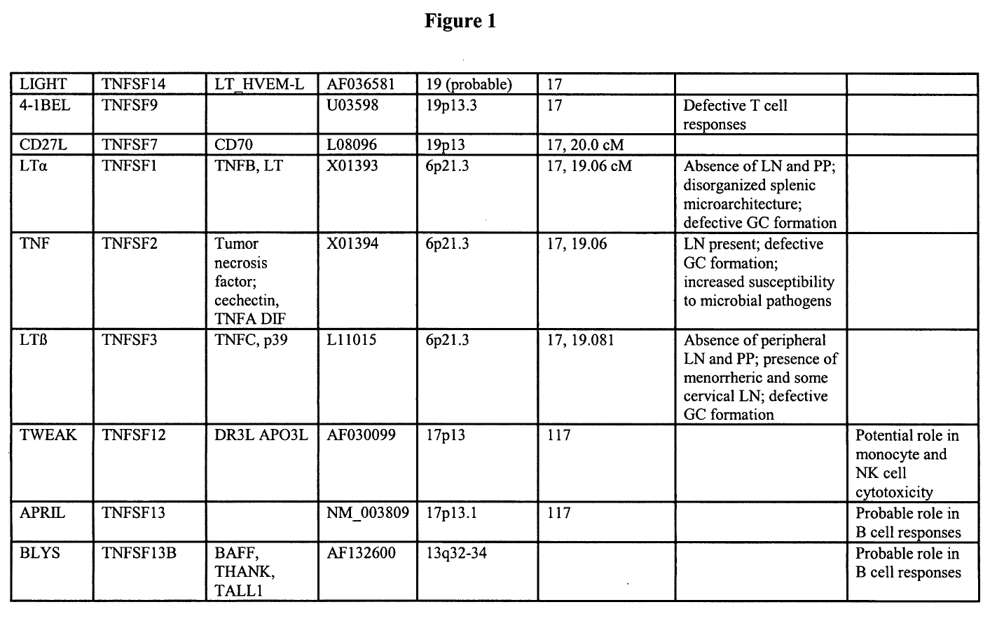

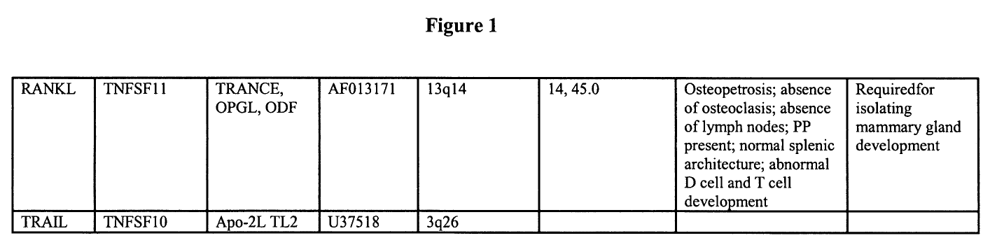

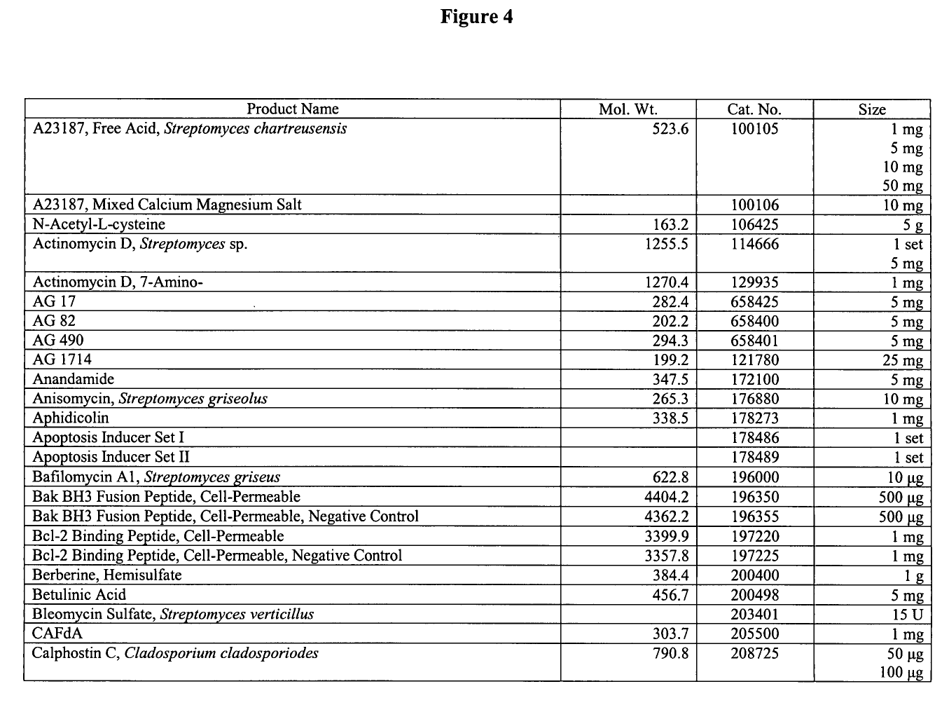

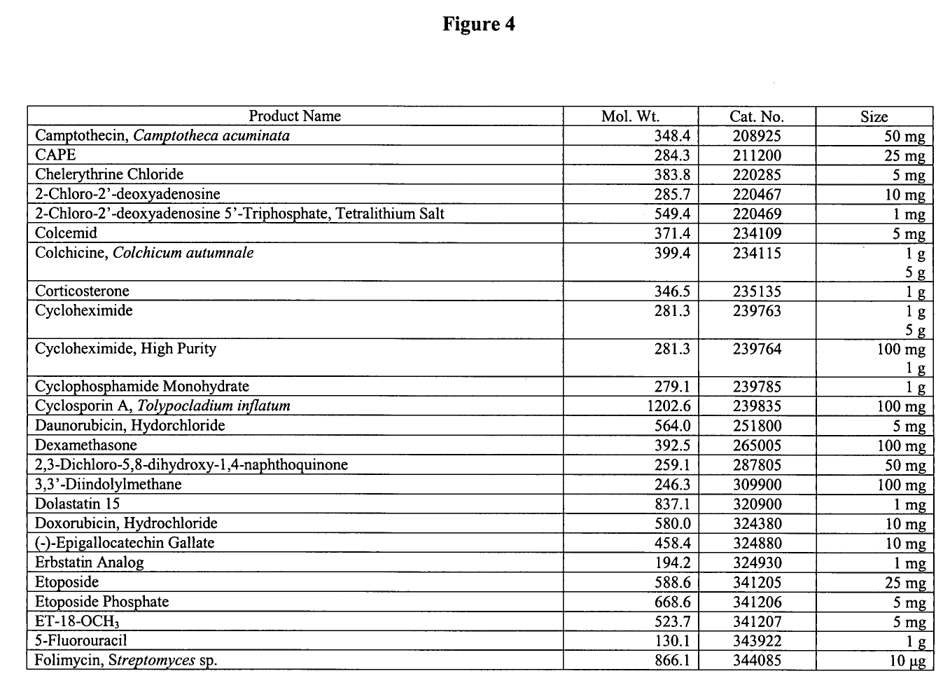

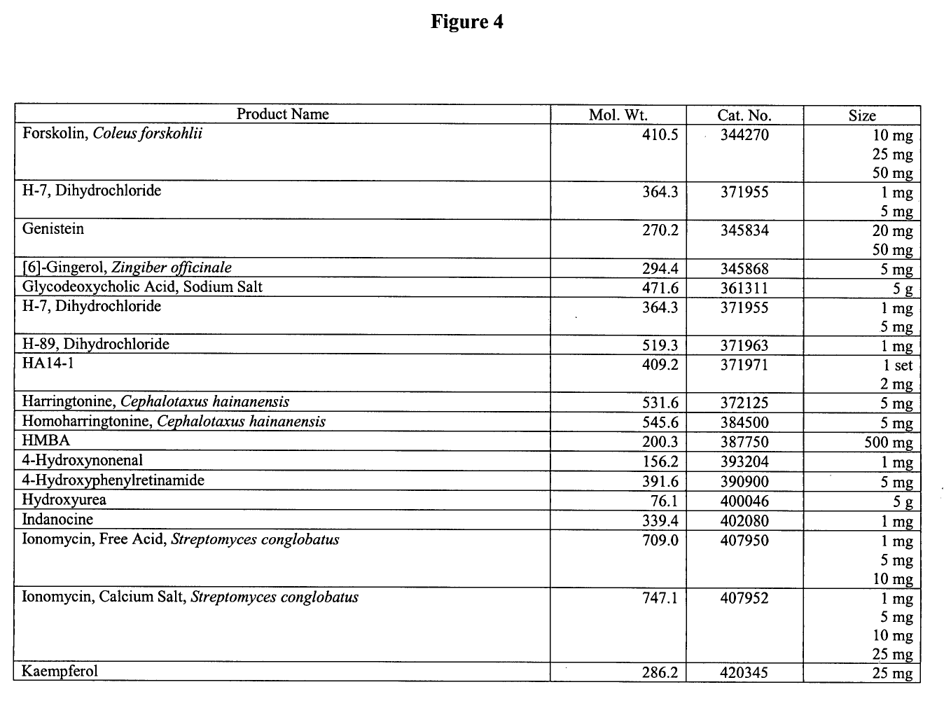

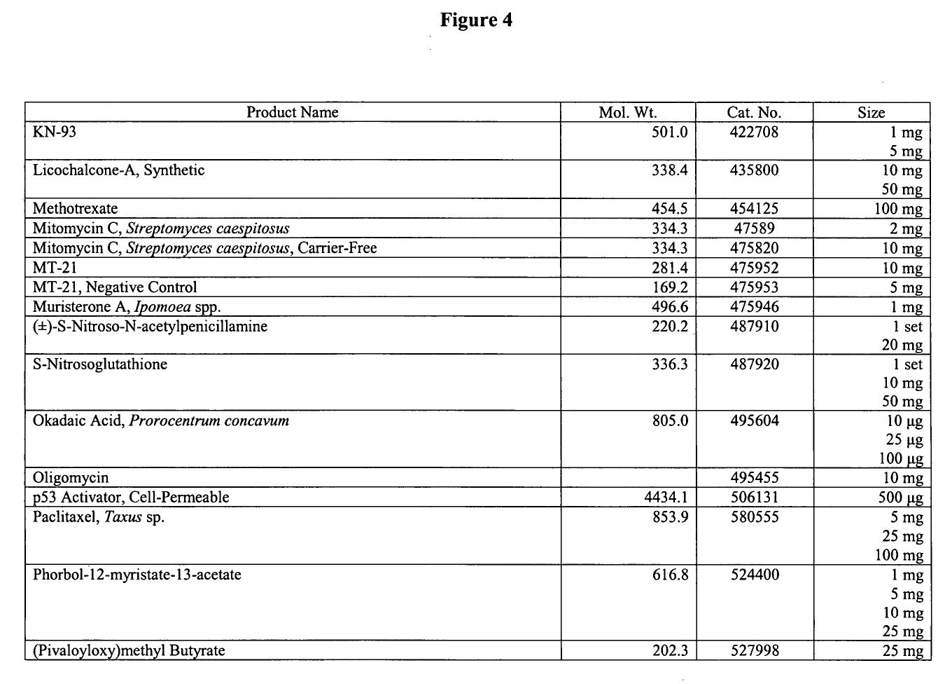

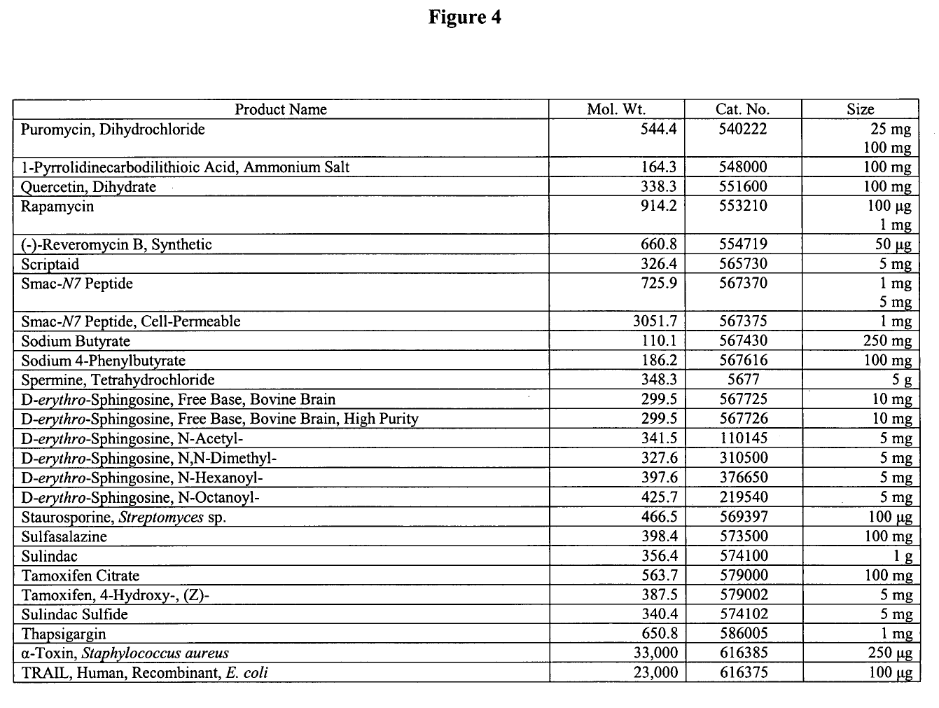

Leukocyte cell viability can be measured by an apoptotic event. Many apoptotic biochemical pathways are known, as are also known agents that act upon those pathways. A list of exemplary death receptors is presented in FIG. 1, whereas in FIG. 4 are presented exemplary compounds that induce cell death. Pathways for apoptosis have also been reviewed by Mak, et al., in Arthritis Res. 4 Suppl 3:S243-52 (2002).

Agents known to inhibit apoptosis can also be used for the identification of compounds that compromise leukocyte cell viability, where compounds that can induce an apoptic event are identified by their inhibition by inhibitors of apoptosis. In one example, a compound screen using an inhibitor of apoptosis includes the following steps:

i) a first blood sample from a mammal with an autoimmune disease, or from a mammal at risk for developing an autoimmune disease, is partitioned into two fractions, one of which is treated with an inhibitor of apoptosis (fraction 1) and the second of which is not treated with the inhibitor (fraction 2),

ii) both fractions 1 and 2 are treated with a test compound and then the viability of the leukocytes contained in the both fractions is measured,

iii) the ratio of viable leukocytes in fraction 1 to the viable leukocytes in fraction 2 is determined,

iv) a second blood sample from another mammal of the same species who does not have, or is not at risk for developing, an autoimmune disease is also partitioned into two fractions, one of which is treated with the inhibitor of apoptosis used in step i) in the same manner as before (fraction 3) and the second of which is not treated with the inhibitor (fraction 4),

v) as before with fractions 1 and 2, fractions 3 and 4 are treated with the test compound and then the viability of the leukocytes contained in the both fractions is measured,

vi) the ratio of viable leukocytes in fraction 3 to the viable leukocytes in fraction 4 is determined.

A compound is determined to have potential therapeutic efficacy if the ratio of step iii) is greater than the ratio of step vi), i.e., a compound is determined to have potential therapeutic efficacy if the samples containing autoimmune leukocytes are protected to a greater extent with inhibitors of apoptosis than those samples that contain leukocytes from non-autoimmune-diseased subjects.

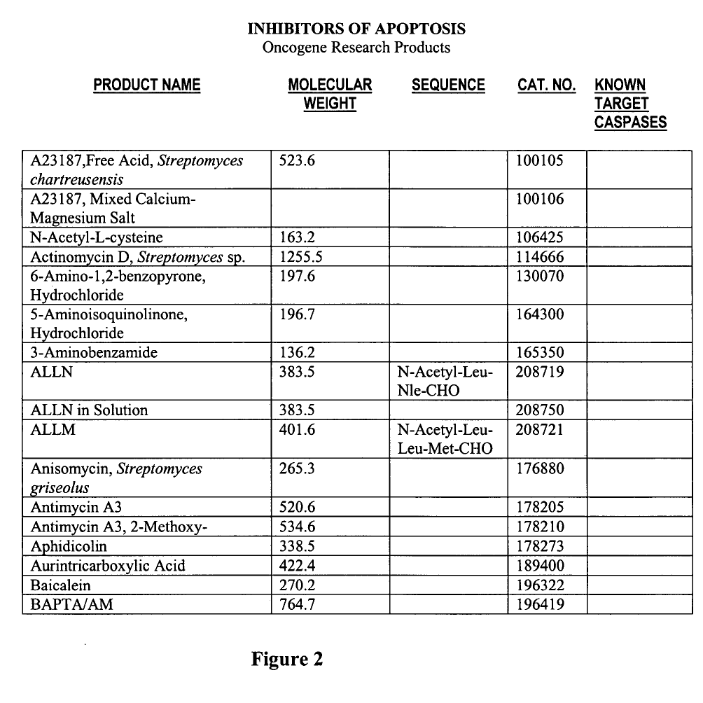

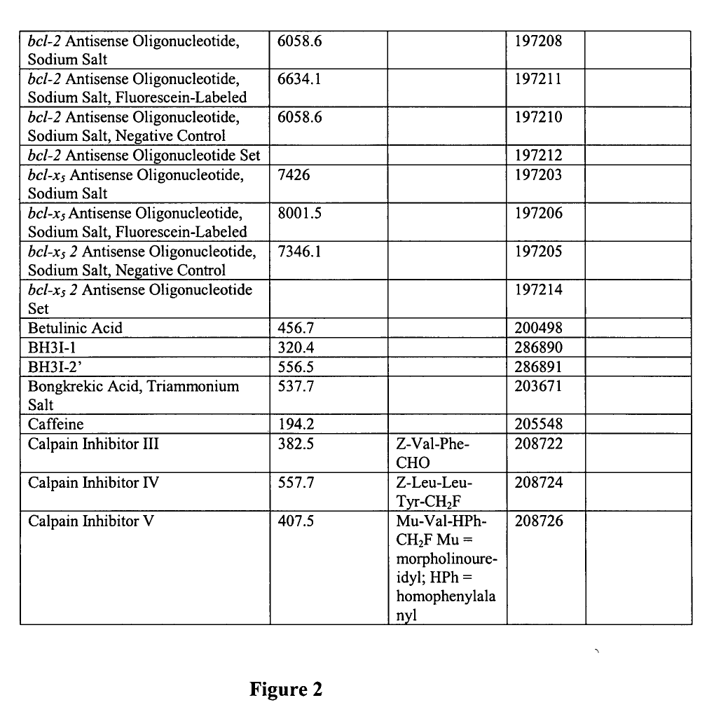

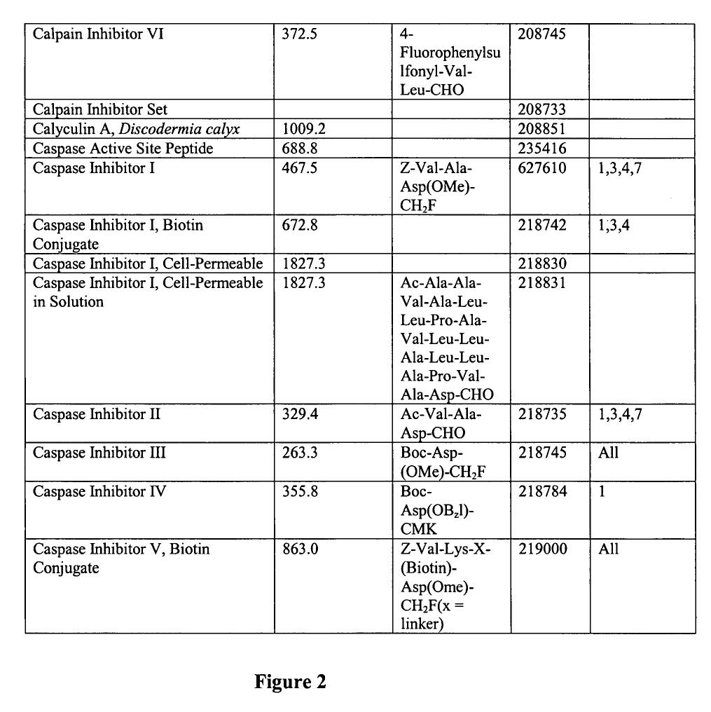

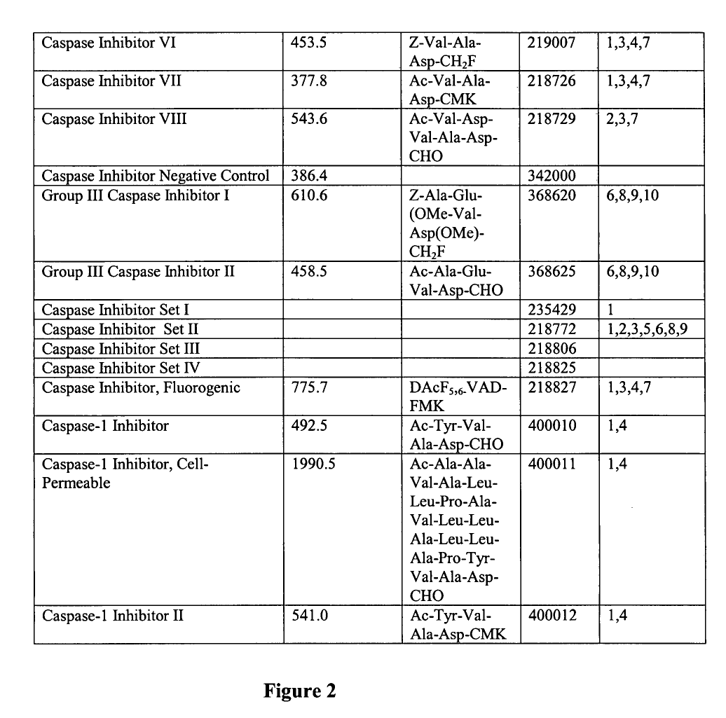

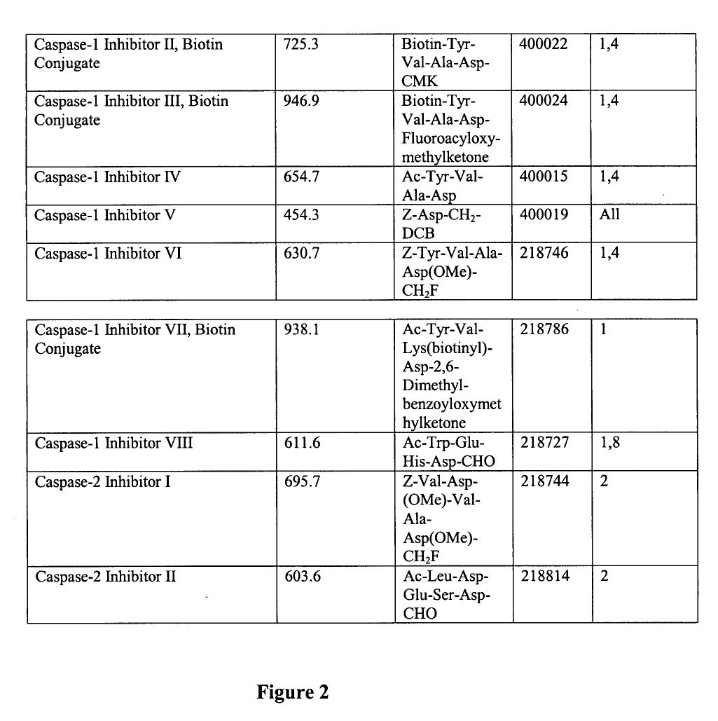

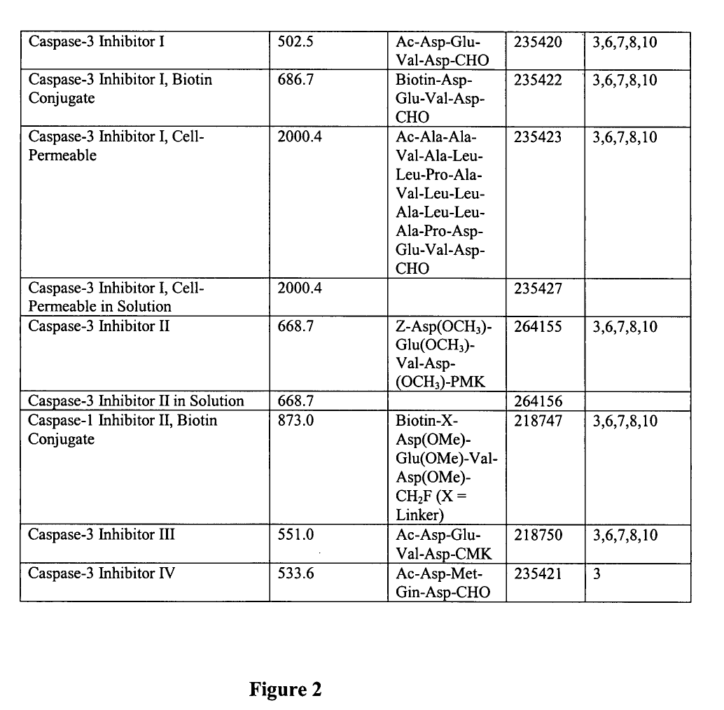

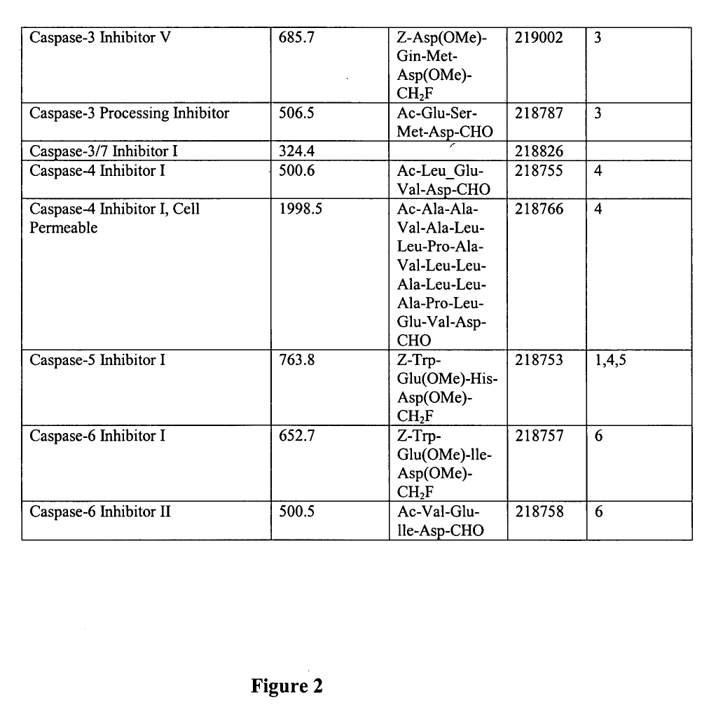

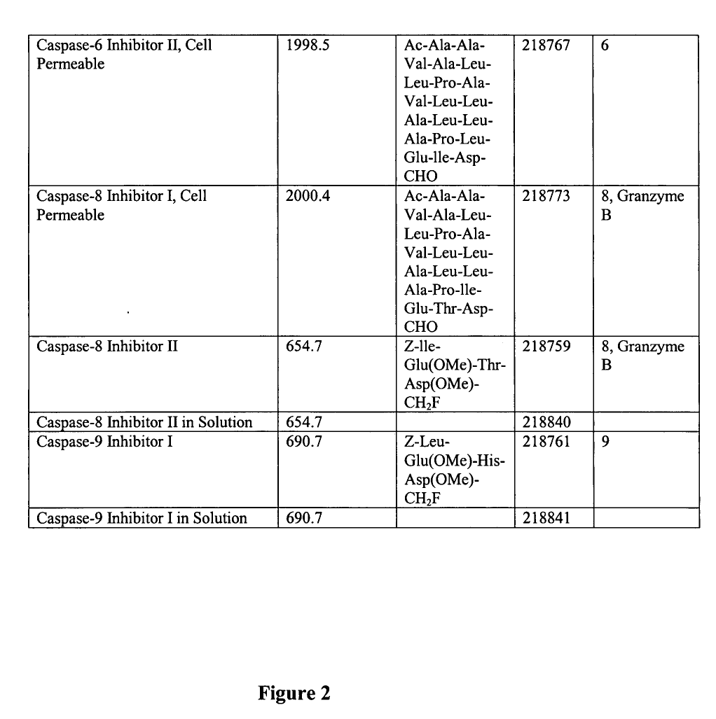

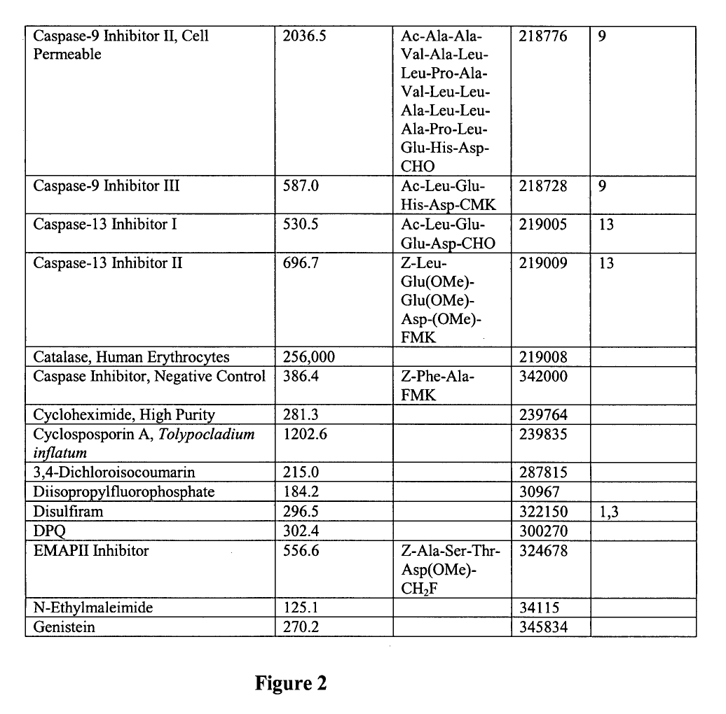

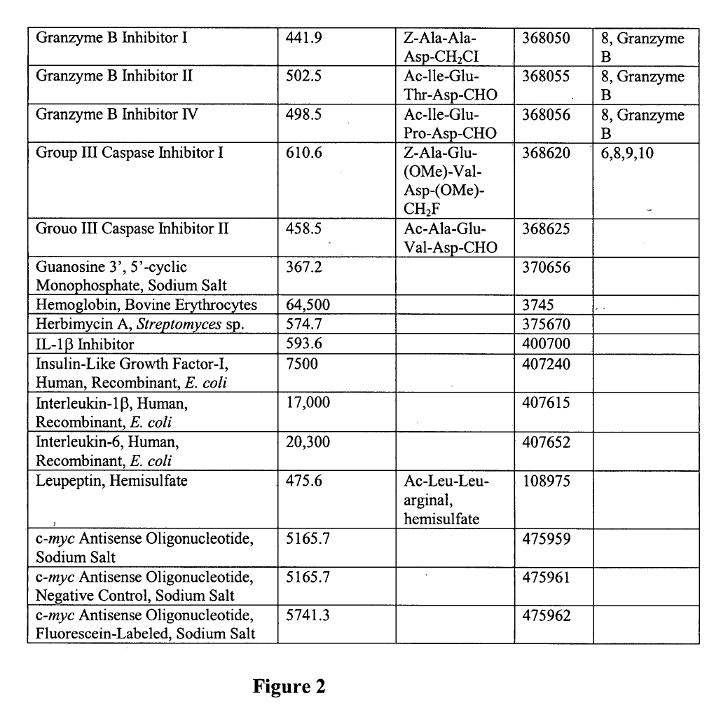

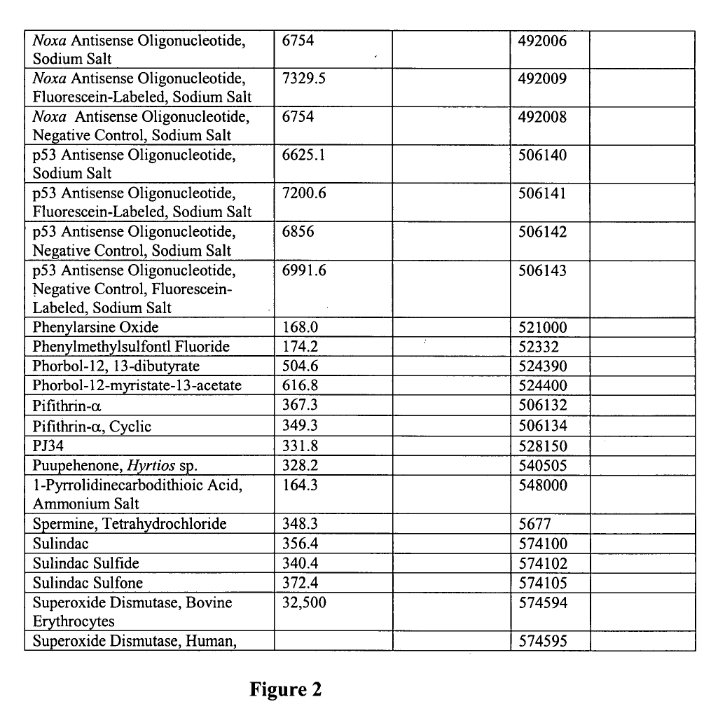

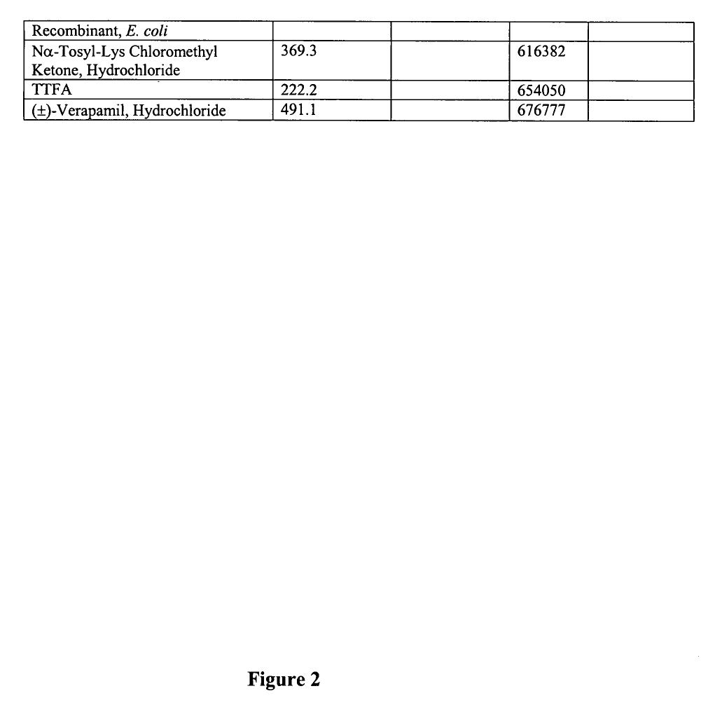

Leukocyte cell viability can also be measured by a necrotic event. Necrotic biochemical pathways are also known to those skilled in the art, as are also known agents that act upon those pathways. Accordingly, a compound screen using inhibitors of necrosis can be used for the identification of compounds that compromise leukocyte cell viability via necrosis-inducing pathways. This screen is similar to that just described for identifying apoptosis-inducing compounds except an inhibitor of necrosis replaces the inhibitor of apoptosis. Therefore, a compound is determined to have potential therapeutic efficacy if the samples containing autoimmune leukocytes are protected to a greater extent with inhibitors of necrosis than those samples that contain leukocytes from non-autoimmune-diseased subjects. Exemplary inhibitors of cell death (through both apoptotic and necrotic pathways) are listed in FIG. 2. An exemplary inhibitor of necrosis is geldanomycin and an exemplary inhibitor of apoptosis is zVAD-fmk.

As described, the invention allows for the identification of compounds that induce cell death or selectively hamper the autoimmune cells by binding to cell surface receptors, cell surface ligands, or by interacting with intracellular proteins which are particular to or over-expressed by autoimmune cells. For example, compounds that stimulate the IL-1 pathway or compounds that interact with converging pathways, such as Fas, FasL, TACI, ATAR, RANK, DR5, DR4, DCR2, DCR1, or DR3, can be identified by the screening methods of the invention.

The methods of the invention can also be used to identify autoimmune cells having the two distinct phenotypes (increased susceptibility to apoptosis or necrosis) described above. In contrast to typical genetic approaches for identifying cells carrying genetic defects, sensitivity to cell death may serve as the initial identification marker. Once cell-death sensitive cells are identified, they can be assessed as to whether they also have the class I antigen presentation defect. Thus, the present invention provides a method of identifying autoimmune cells by (1) assaying the cells for a susceptibility to apoptosis or necrosis and (2) assaying for defects in antigen presentation required for T cell education.

Test Compounds and Extracts

A wide variety of chemical libraries is available in the art and can be screened by the methods of the invention. Compounds capable of treating or preventing autoimmune disease can be identified from large libraries of either natural product or synthetic (or semi-synthetic) extracts, or chemical libraries according to methods known in the art. The precise source of test extracts or compounds is not critical to the screening methods of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed syntheses (e.g., semi-synthesis or total synthesis) of any number of chemical compounds, including, but not limited to, lipid-, saccharide-, peptide-, and nucleic acid-based compounds. Numerous preparations of synthetic compound libraries have been described and many libraries are commercially available. For example, libraries of compound mixtures can be prepared and screened as described by Houghten, et al. in J. Med. Chem. 42:3743-78 (1999). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.).

In addition, those skilled in the art of drug discovery and development readily understand that the methods for dereplication (e.g., taxonomic dereplication, biological dereplication, and chemical dereplication, or any combination thereof) or the elimination of replicates or repeats of materials already known for their ability to treat or prevent autoimmune disease should be employed whenever possible.

When a crude extract is found to induce cell death of autoimmune cells, further fractionation of the positive lead extract is necessary to isolate chemical constituents responsible for the observed effect. Thus, the goal of the extraction, fractionation, and purification process is the careful characterization and identification of a chemical entity within the crude extract that induces cell death. Methods of fractionation and purification of such heterogeneous extracts are known in the art. If desired, compounds shown to be useful agents for treating or preventing autoimmune disease are chemically modified according to methods known in the art.

As described above, assays can be performed to determine whether a particular compound promotes cell death by apoptosis or necrosis. With the selective use of inhibitors of apoptotic events or inhibitors of necrotic events, these methods and be used to determine which protein in a cell death pathway that a compound of the invention modulates. This information can be useful in the development of a therapeutic regime for the amelioration of an auto immune disease.

As described, the screening assays of the invention lend themselves to the discovery of agents or compounds that selectively compromise detrimental autoimmune cells by directly or indirectly activating apoptotic or necrotic pathways. In another example, compounds can be found that further exploit particular receptors on susceptible autoimmune cells by targeting of these receptors with bifunctional conjugates. Such conjugates would be formed by the attachment of toxins or radioisotopes to compounds that are discovered to bind to susceptible cells, thereby potentially augmenting the degree or rate of autoimmune cell depopulation. An example of such an agent is the diphtheria toxin/IL-2 conjugate described in U.S. Pat. No. 5,080,898.

Diagnosing Autoimmune Disease

In another aspect, the invention features a method of diagnosing an autoimmune disease, or a predisposition to an autoimmune disease, in a mammal, preferably a human, by contacting a blood sample from the mammal with a compound that preferentially decreases the cell viability of leukocytes followed by measuring leukocyte viability, wherein a decrease in viability is indicative of the mammal having an autoimmune disease or a predisposition to an autoimmune disease. Leukocyte viability is measured relative to a control sample of leukocytes obtained from a mammal of the same species not having and not at risk for developing an autoimmune disease, with the control sample also being treated with the compound under the same conditions used to test the sample from the mammal being diagnosed.

As described above for the screening assays of the invention, instead of leukocytes from a non-autoimmune-diseased control subject, control substances can include a second blood element, obtained from the autoimmune-diseased mammal, which is not susceptible to cell death via an aberrant apoptosis or necrosis pathway inherent to autoimmune cells. Desirable examples include non-autoimmune leukocytes or erythrocytes.

Leukocytes that have suffered an apoptotic or necrotic event can be quantitated by known flow cytometric methods, described, for example, by Darzynkiewicz, et al., in Clin. Lab. Med. 21:857-73 (2001). Laakko, et al., in J. Immunol. Methods 261:129-39 (2002) have recently described the use of merocyanine 540, which binds to disordered membranes, in the flow cytometric detection of apoptosis in human and murine cells. In addition, Prieto, et al., in Cytometry 48:185-93 (2002) have described using a flow cytometric analysis to determine “apoptotic rate” as an indicator for the quantification of apoptosis in cell culture. Some methods for the measurement of leukocyte apoptosis require the removal and or lysis of erythrocytes as part of the procedure, necessitating the partitioning of samples for accurate erythrocyte and leukocyte quantitation. Other techniques rely on markers that are cell membrane expressed, and therefore do not require cell lysis.

In methods of diagnosing an autoimmune disease, autoimmune cell viability can also be measured relative to a second blood element in the sample, wherein a decreased ratio of viable autoimmune cells relative to the second blood element versus the same ratio found in a comparable sample not contacted with the compound is indicative of autoimmune disease. In one example, the diagnostic screen is an in vitro assay and the second blood element used for comparison purposes is a non-autoimmune leukocyte. In this example, a sample is obtained from apportioned into two fractions and one fraction is treated with the compound and the other is not. The samples are incubated for an appropriate amount of time, preferably between 12 and 36 hours, and both are evaluated for viable autoimmune leukocytes and viable non-autoimmune leukocytes. Alternatively, the second blood element can be an erythrocyte. Flow cytometric techniques for erythrocyte quantitation are known, as described by Davis in Clin. Lab. Med. 21:829-40 (2001).

In another example in which a second blood element is used as a control cell for determining the decrease of susceptible autoimmune cells in the diagnosis of an autoimmune disease, a first blood sample is withdrawn from a subject before administration of a compound known to decrease the viability of autoimmune cells and the ratio of viable autoimmune cells to the second blood element is measured. The compound is then administered to the subject and, after a period of time, desirably 6 to 24 hours, a second blood sample is withdrawn from the subject and the ratio of viable autoimmune cells to second blood element is measured. If the period of time between compound administration and withdrawal of the second blood sample is sufficient for measurable autoimmune cell death to occur, the level of autoimmune cell depopulation can be determined by a cell population count of the autoimmune leukocytes and a ratio determined using the second blood element. Alternatively, if the second blood sample is withdrawn before appreciable autoimmune cell death takes place, but after the initiation of measurable apoptotic or necrotic events, techniques useful for the measurement of necrosis or apoptosis, such as those referred to above for the screening assay, can be used for the analysis of viable autoimmune cells. A diagnosis of autoimmunity or predisposition of autoimmunity is rendered if the ratio of viable autoimmune cells to the second blood element in the second blood sample is less than that ratio in the first blood sample. If a time course for the decrease of autoimmune cell viability is desired, the diagnostic method just described can be employed with multiple blood sample withdrawals (e.g. over 24 hours, 48 hours, 72 hours, or 1 week) from the subject after compound administration, analyzing the ratio of viable autoimmune cells to second blood element in each sample.

As described above, the diagnostic methods of the invention can complement a therapy which includes the administration of a compound or a mixture of compounds that selectively depopulate leukocytes, particularly autoimmune cells.

Monitoring Treatment of Autoimmune Disease

For autoimmune therapies that include selectively decreasing the viability of leukocytes, desirably those leukocytes involved in an autoimmune response, by apoptotic and/or necrotic processes, another aspect of the invention features a method for the monitoring a therapy for a human patient with an autoimmune disease or a predisposition to said disease. The method includes the steps of contacting a blood sample from the patient with a compound that preferentially decreases the viability of leukocytes, followed by measuring leukocyte viability, wherein the decrease in viability is indicative of the efficacy of the therapy. Leukocyte viability can be measured relative to a control sample of leukocytes obtained from a person not having and not at risk for developing an autoimmune disease, with the control sample also being treated with the compound under the same conditions used to test the sample from the patient being diagnosed.

Desirably, the leukocytes that are monitored are those defective in protection from apoptotic cell death by stimulus, for example, of the TNF-alpha receptor, IL-1beta receptor, IL-6 receptor, IL-12 receptor, IFN-gamma receptor, CD40, CD40, CD28, Fas, or necrotic cell death by any stimulus.

In another aspect, autoimmune cell viability is measured relative to a blood element in the sample, wherein the decrease in the ratio of viable autoimmune cells versus those found in an identical sample not contacted with the compound is indicative of the efficacy of the therapeutic treatment. In one example, the diagnostic screen is an in vitro assay in which non-autoimmune leukocytes are the blood element used for comparison purposes, wherein a sample is apportioned into two fractions and one fraction is treated with the compound and the other is not. The samples are incubated for an appropriate amount of time, preferably between 12 and 36 hours, and the viability autoimmune cells and non-autoimmune leukocytes in both fractions is evaluated.

In another example, a control blood sample is withdrawn from the patient before administration of a compound or compound mixture known to selectively compromise autoimmune cells. The ratio of viable autoimmune cells to non-autoimmune leukocytes is measured in this control blood sample and a compound or compound mixture is administered to the subject that is known to reduce the viability of autoimmune cells. After a period of time, a second blood sample is withdrawn from the subject and the ratio of viable autoimmune cells to non-autoimmune leukocytes is also measured. If the period of time between compound administration and withdrawal of the second blood sample is sufficient for measurable autoimmune cell death to occur, the level of autoimmune cell depopulation can be determined by simple cell counts of autoimmune cells and non-autoimmune leukocytes and compared to those same cell counts observed in the control sample. Alternatively, if the second blood sample is withdrawn before appreciable autoimmune cell death takes place, but after the initiation of measurable apoptotic or necrotic events, techniques that are used for the measurement of necrosis or apoptosis, such as those referred to above for the screening assay, can be used for the analysis of viable autoimmune cells in both the first and second sample. The efficaciousness of the therapy is determined by the level of viable autoimmune cells observed, with an efficacious therapy resulting in a lower ratio of viable autoimmune cells to non-autoimmune leukocytes. This monitoring method can be employed with multiple sample withdrawals after compound administration, analyzing the ratio of compromised autoimmune cells to non-autoimmune leukocytes for each sample and adjusting the therapeutic regimen accordingly.

Such monitoring methods can make it possible to measure the effect of particular doses agents on the apoptosis of autoimmune cells concurrently with therapeutic treatment of an autoimmune individual, where such a treatment includes the depopulation of autoimmune cells. Moreover, such a monitoring system can enable dose optimization or adjustment of the dose for maximizing autoimmune cell death while minimizing exposure of the individual to toxic doses of the therapeutic agent.

The assays of the invention can thus be used to tailor autoimmune therapy to the needs of a particular individual. For example, a treatment regimen that features the induction of TNF-alpha, as mentioned above, can be carried out every day or every other day in order to measure the therapy's effect on autoimmune cell death rate in a manner that adjustment to the administered dose, duration of treatment (i.e., the period of time over which the patient will receive the treatment), or treatment regimen (i.e., how many times the treatment will be administered to the patient) of treatment agents and can be adjusted to optimize the effect of TNF-alpha inducement on autoimmune cell death and minimize the exposure of the patient to these TNF-alpha-inducing agents. Of course, the skilled artisan will appreciate that the assay can be performed at any time deemed necessary to assess the effect of a particular regimen of TNF-alpha induction therapy on a particular individual (e.g., during remission of disease or in a pre-autoimmune individual). The ability for such a monitoring system can allow for the administration of agents over a longer course of treatment, preferably over a period of months, more preferably over a period of years, and most preferably over a lifetime, than was previously possible.

The present monitoring system can also be used to identify new doses, durations of treatment, and treatment regimens for apotosis- or necrosis-inducing agents that were previously discounted for being useful as treatments because there was no effective way to monitor their effect. In one example, researchers failed to identify a therapeutic dose of BCG, a TNF-alpha inducing agent, for the treatment of Type I diabetes due to the lack of methods for monitoring the effect of BCG in vivo [Allen et al., Diabetes Care 22:1703 (1999); Graves et al., Diabetes Care 22:1694 (1999)]. The present methods address this need.

Methods of the invention can be used to identify a dose, a duration of treatment, or a treatment regimen of agents that depopulate selected populations of leukocytes for the reduction or elimination of side effects associated with a particular autoimmune disease. It is often the case that the side effects associated with an autoimmune reaction are responsible for mortality of autoimmune patients. Methods of the invention can make it possible to eliminate the symptoms associated with, for example, vascular collapse associated with diabetes, blindness or kidney failure associated with Type I diabetes, or skin eruptions associated with lupus.

Most desirably, the monitoring system of the present invention identifies how agents that induce apoptosis/necrosis can be used in a treatment regimen that prevents disease progression or even halts disease in a patient diagnosed with an autoimmune disease.

For the any of the methods of the invention, the autoimmune disease can be Alopecia, Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjogren's Syndrome, Stiff-Man Syndrome, Takayasu Arteritis, Temporal Arteritis/Giant Cell Arteritis, Ulcerative Colitis, Uveitis, Vasculitis, Vitiligo, Wegener's Granulomatosis, or myasthenia gravis.

Equipment

For screening libraries of compounds using the methods of the present invention, an automated workstation can be used. One example is the Biomek® FX workstation (Beckman-Coulter) integrated into a SAGIANTM core system. This system includes multichanneled pipetting for rapid compound transfers and reagent addition and eight-channel independent axis pipetting for adding control samples and performing dilutions. Method creation and schedule optimization can be performed using SAMI® software.

A Blood Lymphocyte Cell Preparation Center can be used for repeat analysis of patient blood samples for sensitivity to inducers of cell death. The PrepPlus Workstation removes small amounts of blood from vacuum tubes and places it directly in 96 or 394 well plates, and the TQ Workstation serially adds RBC lysing reagents to obtain separated lymphocytes already located in the 96 or 394 well plates. The LJL Analysis HT system and Biomek system represents the robotic arms that shuttles the plates along the assembly line and contains the programmable fluid dispensing equipment and computer software.

High-output platform cellular screening involves, e.g., 96 or 394 well plates containing 1000 to 5000 cells per well that are read by a multi-parameter fluorescent reading machine that analyzes the cells without their removal from the plate. For example, the Cellomics Kinetic Array Scanner II is a cell scanner with up to eight fluorescent probes for detecting labeled, living cells contained in each well. These fluorescent probes can detect cell surface markers, intracellular constituents, and apoptotic markers. This automated technology is rapid with speeds up to one well per 0.5 second and leaves cells undisturbed in the well, thereby permitting reanalysis at frequent intervals. The apparatus can be set up in a temperature controlled chamber, thus accurately assessing the kinetics of ongoing cell death as well as the specific tracking of cell death among aminor subpopulation.

Flow cytometry can be performed using a Facscalibur flow cytometer (BD Biosciences, San Jose, Calif.). Samples were incubated for 18-24 hours in the absence or presence of TNF-α (20-50 ng/mL), after which apoptotis can be determined by direct detection using FITC-conjugated annexin V and Propidium Iodide (PI) from R&D Systems (TACS Annexin V-FITC Apoptosis Detection Kit; Minneapolis, Minn.). Early apoptotic cells were defined as positive for annexin V only, whereas late apoptotic cells were defined as being positive for both PI and annexin V.

Splenocyte Isolation

Female NOD mice were from Taconic Farms (Germantown, N.Y., USA) and C57BL/6J (B6) mice from The Jackson Laboratory (Bar Harbor, Me., USA). The NOD mice were maintained under pathogen-free conditions and screened for the onset of diabetes by monitoring body weight and blood glucose; they were diagnosed as diabetic when two consecutive blood glucose concentrations exceeded 400 mg/dL. The C57BL/6J mice were housed under normal conditions.

In order to obtain high viability for those assays using splenocytes, gentle treatment throughout the isolation procedure is important. Optimized results were obtained as follows: a mouse is sacrificed by cervical dislocation and the spleen removed via an abdominal incision. The spleen is then gently inflated with several mLs of RPMI/10% FBS using a 22 gauge needle. Any fluid leaking out of the spleen is collected as it contains splenocytes. A small opening is created on one end of the spleen and the splenic tissue expelled through this opening with a “scraping” motion using blunt tipped forceps. The resulting clumps of tissue are homogenized by repeated gentle pipetting. The crude splenocyte preparation is then filtered through a 40 micron mesh filter.

If desired, red blood cells (RBCs) can be removed from the crude splenocytes preparation by one of the following three methods:

i) Splenocytes can be resuspended in 10 mL of NH₄Cl lysis buffer (140 mM NH₄Cl in 17 mm Tris buffer, pH 7.65) and incubated for 10 min at room temperature, followed by washing once with RPMI/10% FBS,

ii) A 5 mL suspension of splenocytes in RPMI/10% FBS can be layered on top of 5 mL of Ficoll-Paque Plus (Amersham Biosciences AB, Uppsala, Sweden) in a 15 mL centrifuge conical tube. The tubes are then centrifuged at 4° C. for 10 min at 1300×G and the buffy coat on the ficoll/RPMI interface collected. The cells are then washed once with 10 mL RPMI/10% FBS.

iii) A population of about 5×10⁷ splenocytes and RBC's in RPMI/5% FBS is incubated with Terl 19 MACS beads (Miltenyi Biotec, Auburn, Calif.) for 15 min at 4° C., washed once, and passed over a LS column (Miltenyi Biotec), which retains the RBCs in the sample. The purified splenocytes are eluted from the column and washed once with RPMI/10% FBS. This method of selecting for (or against) cells that express receptors or ligands on the cell surface and purifying (or removing) the selected cells is a general one and can be applied to any cell subtype, where the subtype is characteristic of a cell-surface-expressed protein (see U.S. Pat. Nos. 4,452,773 and 6,576,428).

### EXAMPLE 1

### Fractionation and Sensitivity Studies on Murine Splenocytes

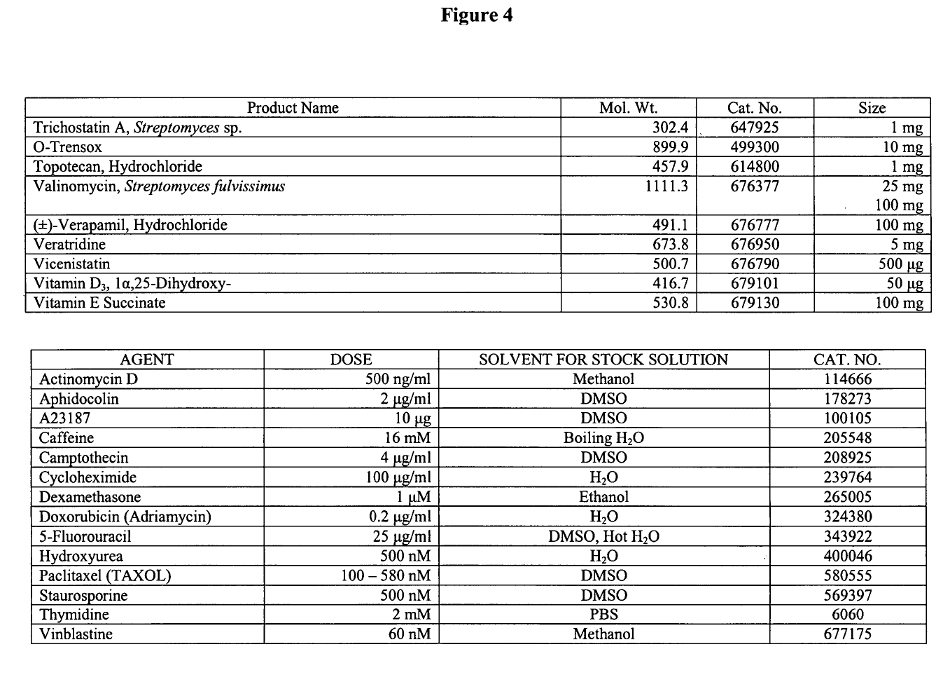

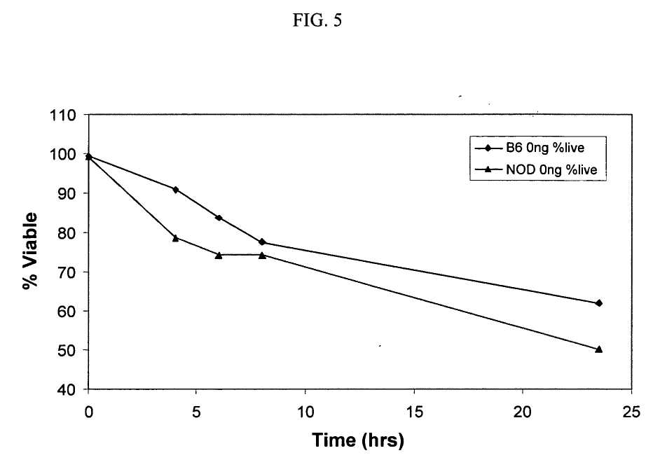

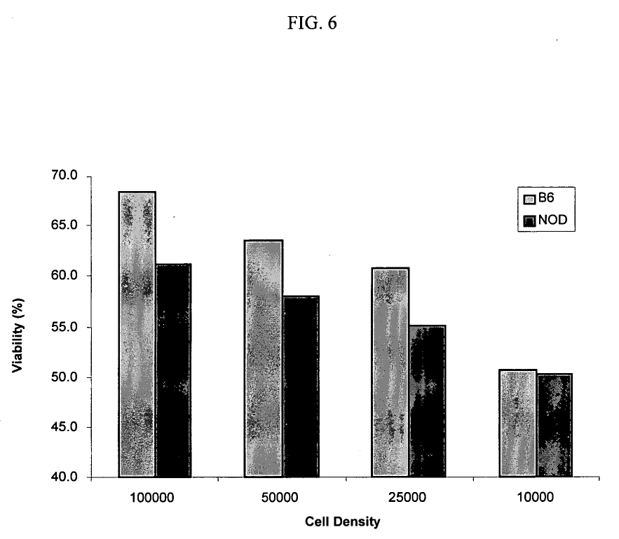

It is known that freshly prepared, unstimulated splenocytes die rapidly in culture. This is illustrated in FIG. 5, where the quantified death of splenocytes that were isolated from the spleens of C57BL/6 and of pre-diabetic NOD mice were studied during regular tissue culture at 37° C. and 95% humidity in RPMI media supplemented with 10% FBS and antibiotics. After splenocytes isolation, RBCs were removed using a Ficoll gradient. The splenocytes were cultured at a density of 5×10⁶ splenocytes per well (100 μL per well) in flat-bottomed 96 well plates. Samples were taken at 0, 4, 6, 8, and 24 hours and the viabilities of the splenocytes were determined using trypan blue dye. As shown in FIG. 1, a steady decline of viable cells in culture is observed, with the viability of the NOD splenocytes lower than that of B6 splenocytes. With repetition of these studies at different cell culture densities, from 5×10⁵ to 5×10⁷ cells/mL, a trend was observed (see FIG. 6). It was found that the average viability of the NOD splenocytes at all densities tested tended to be lower as compared to their B6 counter parts, with the exception of very low densities. At very low cell densities, where cell death is greater than 50%, the NOD accelerated death effect is lost.

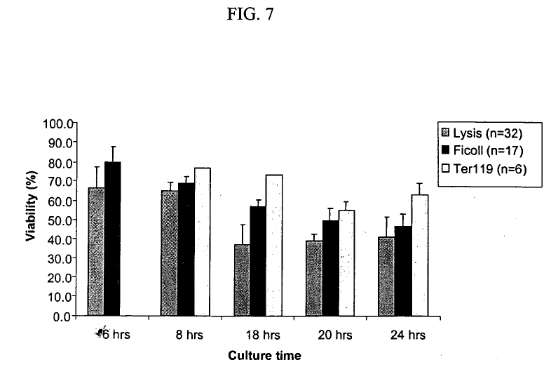

In vitro splenocyte viability is also dependent upon removal of red blood cells. FIG. 7 shows the results of splenocyte-viability for the three methods of RBC removal used in the methods of the invention. Using flow cytometry with Anenxin V and PI, the compared percentage of apoptotic cells and cell viabilities over time during culture of splenocytes that were purified using either NH₄Cl lysis (n=32, where n is the number of splenocyte samples), ficoll-paque (n=17), or Ter119 MACS beads (n=6) were studied. Ter119 is a cell surface marker that is expressed on mature mouse erythrocytes and erythroid precursor mouse cells, but not on lymphoid or myeloid cells. The rate at which splenocytes underwent spontaneous apoptosis was clearly lowest in the preparation that was purified using Ter119 magnetic beads (at 24 hrs culture, p=0.00006 versus ficoll and p=5×10⁻⁹ versus NH₄Cl in a One-Tailed Student's T test), and highest in the sample in which the red blood cells were lysed using NH₄Cl (p=0.005 versus ficoll). The use of Ter119 magnetic separation to remove the RBCs therefore maximizes the ability to monitor selective cell death specific to autoimmunity.

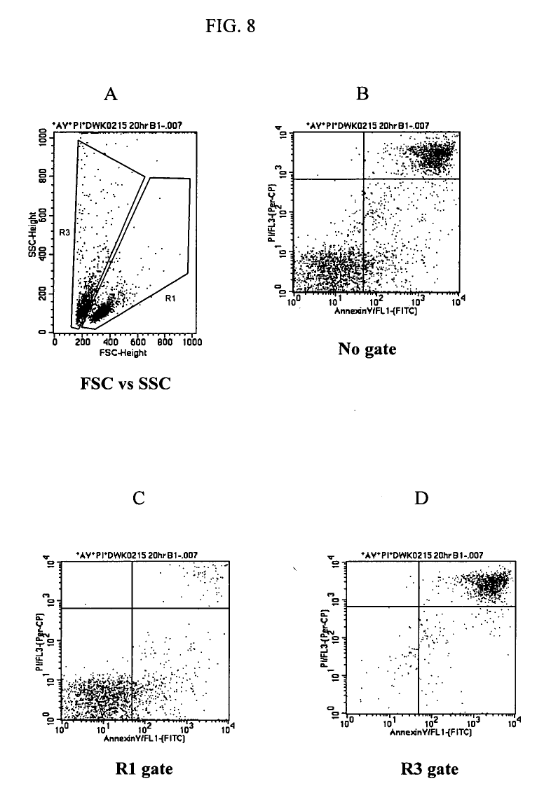

The identification of dying/dead cells can be optimized by locating live and dead cell populations on Forward Scatter (FSC) versus Side Scatter (SSC) plots by flow cytometry. Ter119 purified B6 splenocytes were cultured for 20 hrs, stained with Annexin V and PI, and analyzed on by flow cytometry (FIG. 8A). The cells show up in two main populations on FSC vs SSC. The ungated, Annexin V versus PI plot (FIG. 8B) showed a typical distribution of cells that remained unstained (live) and cells that stained with both Annexin V and PI (late apoptotic/dead). Studies were also performed that gated the Annexin V/PI plot using regions R1 and R3 (FIGS. 8C and 8D, respectively). The results show that the majority of late apoptotic/dead cells were found in the R3 region, whereas the live cells originated in the R1 region. This was as expected, since cells shrink and become irregular during apoptosis, resulting in a decrease of the Forward Scatter and an increase of the Side Scatter. Thus, by gating on R1 or R3, one can “tune” the apoptosis assay to look at a predominantly live or predominantly dead cell population.

The spleen contains a variety of different immune cells. These can be distinguished further into T-cells (mostly CD4 and CD8) and non-T-cells (mostly B-cells and macrophages). Magnetic separation methods were used to sort the splenocytes into T-cell and non-T-cell fractions. For the isolation of T-cells, Miltenyi Biotec's Pan T magnetic bead cocktail were used. This cocktail contains antibodies against CD11b, CD45R, DX5, and Ter-119. As a result, all Non-T cells, i.e. B cells, NK cells, dendritic cells, macrophages, granulocytes, and erythroid cells; are depleted from the sample, leaving only T-cells. For the isolation of non-T-cells, a mixture of magnetic beads directed against CD90 and Ter-119 were used. This depletes all T-cells and erythroid cells, leaving only the non-T cells.

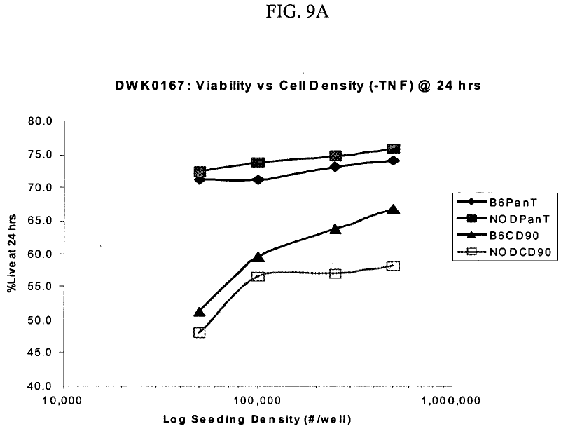

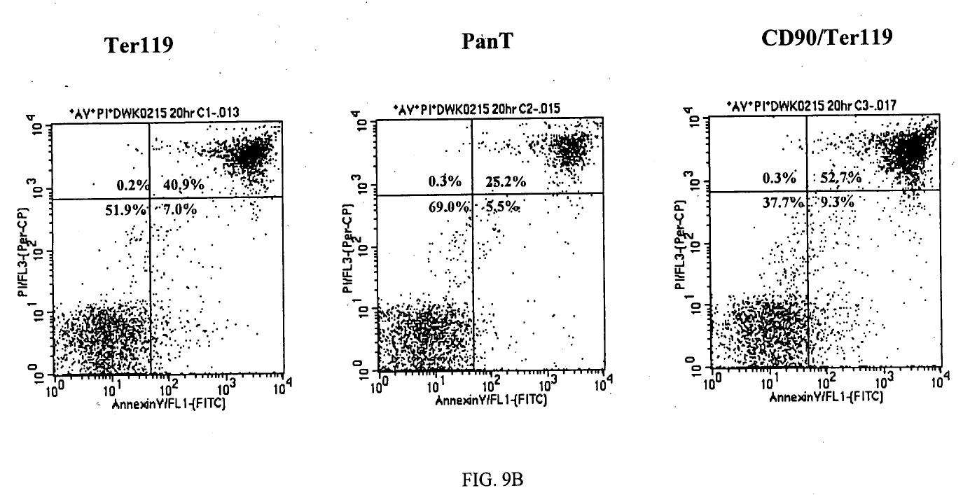

The separate populations of non-activated T-cells and non-T-cells from both C57B1/6 and NOD mice were cultured and cell viability at various cell densities after 24 hrs were determined (FIG. 9A). There was a clear difference in viability between the T-cell and the Non-T-cell fractions. While the viability of the T-cell fractions from both B6 and NOD mice (purified by using a PanT magnetic column) were relatively independent of cell density, the viability of the non-T-cell fractions (purified by using a CD90 magnetic column) decreased considerably when the cells were cultured at low seeding densities. As previously observed for non-fractionated splenocytes, the NOD non-T-cells had a reduced viability as compared to their B6 counterparts. Thus, the decrease in viability that is observed previously for non-fractionated cultured splenocytes is attributed mainly to the non-T-cell population.

FIG. 9B illustrates how the viabilities T and non-T sub populations combine in an intermediate viability for the complete preparation. In this example, purified splenocytes from a C57B1/6 mouse were purified in three different ways to leave a mix of both T cells and non-T cells (purified by using only a Ter-119 column); only T cells (purified by using a PanT magnetic column); or only Non-T cells (purified using CD90+ Ter-119 columns). After 20 hours culture, the viability of the different preparations was determined using PI/Annexin V flow cytometry. The viability results observed were 69.0% for the T cells and 37.7% for the non-T cells, with the viability of the mix of T cells and Non-T cells (51.9%) approximately half-way between these two.

The T and non-T cell fractions were further fractionated into CD4/CD8 T cells and CD11B (Monocytes and Macrophages)/CD19 (B cells) cells for both NOD and B6 mice. As shown in FIG. 13, the NOD T cell population with greatest TNF sensitivity is CD8⁺. NOD CD4⁺ T cells have overall decreased survival relative to their normal B6 counterparts but are not TNF sensitive. These data demonstrate that the accelerated death of autoreactive lymphoid cells is different depending upon the cell population of origin. The data also demonstrate that different cell populations can be isolated for different screening assays, with appropriate fine tuning of the assays of the invention possible.

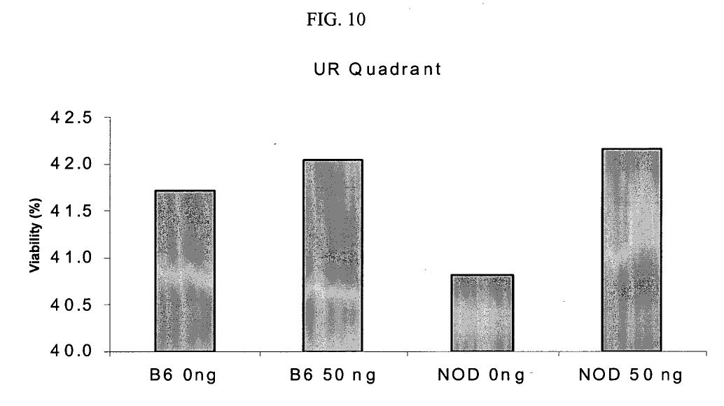

The difference in sensitivity between B6 and NOD splenocytes to TNF-α was then examined. B6 and NOD splenocytes were plated out in U-bottom shaped 96 well plates at a density of 25,000 cells per well in 100 μL of medium, with and without 50 ng/mL TNF-α added. The cells were cultured for 24 hrs, stained with Annexin V and PI, and analyzed by flow cytometry (FIG. 10). It was observed that NOD splenocytes were much more sensitive to TNF-α compared to the B6 control splenocytes, confirming that the NOD splenocyte population contains a select fraction of cells that show TNF-α sensitive apoptosis.

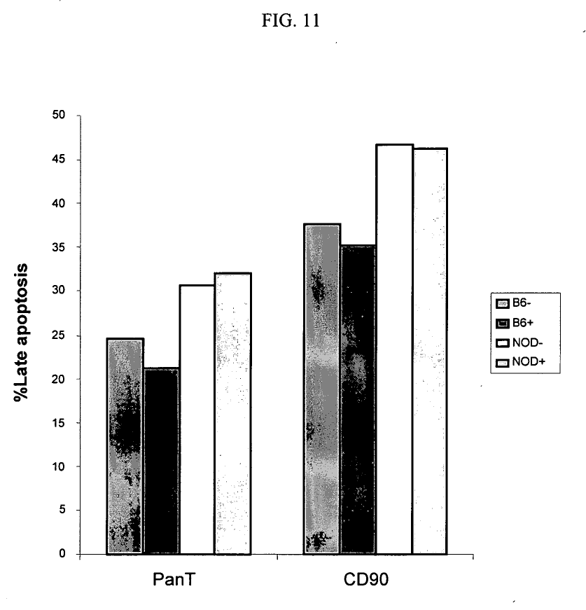

After culture at 37° C. for 26 hours, the cell death of NOD vs. B6 T cells and NOD vs. B6 non T cells was quantified by AnnexinV and PI (FIG. 11). Late death is shown for cells obtained from a single NOD and a single B6 mouse. The B6 T cells did not show TNF-α sensitivity while B6 T cells with TNF showed increased viability, an expected protective response to TNF-α. An opposite response was observed with NOD T cells, which showed increased death, which was further accelerated upon exposure of the cells to TNF-α.

Samples obtained from a single NOD and a single B6 mouse were also examined for cell death by PI staining alone (FIG. 12) after culturing at 32° C. for 18 hours. As before, these data show that B6 T cells do not exhibit TNF induced death, with the opposite result observed in NOD T cells where an exposure to TNF-α results in death of a subpopulation of T cells. In addition, it was found that under the conditions of 32° C. culture for 18 hours, NOD T cells have greater viability and display greater sensitivity to TNF-α.

The difference in viability between CD4 and CD8 T cells was also examined (FIG. 13).

### EXAMPLE 2

### Assay for the Diagnosis of Autoimmune Disease.

Initial Assays for Cell Death in Human PBLs

In an initial set of assays for characterizing the response of PBLs from human diabetics and control subjects to incubation with TNF-α or other candidate compounds in vitro, uptake of the dye Trypan Blue, a relatively nonspecific measure of cell death, was used. Human PBLs from non-diabetic subjects had a 0-10% level of Trypan Blue uptake when examined immediately after isolation and had an up to 20% level of uptake after 24 hours in culture. Exposure of the cells to TNF-α (20 ng/mL) for 24 hours did not significantly alter the extent of Trypan Blue uptake by the PBLs from a small cohort of non-diabetic subjects (FIG. 14B). However, under the same conditions, a consistent increase in Trypan Blue uptake in the PBLs from Type I diabetics was observed (FIG. 14A), with TNF-α further increasing Trypan Blue uptake in Type I diabetic PBLs to various degrees. The extent of TNF-α induced in Trypan Blue uptake by PBLs from diabetics was not significantly related to patient age (FIG. 14C) or duration of diabetes. However, there appeared to be an inverse correlation between the age-at-onset and the extent of the TNF-α induced-increase in Trypan Blue uptake by the PBLs (FIG. 14C). In general, the most robust in vitro cell death responses to TNF-α were observed in PBLs from those Type I diabetics with the earliest age-of-onset. This result is attributed to the increased severity of a defect underlying the enhanced susceptibility to TNF-α-induced apoptosis, which in the NOD mouse appears to be contributory to the pathogenesis of beta cell autoimmunity, and which is most severe in human Type I diabetics, whose disease is penetrant at an early age.

Although increased Trypan Blue uptake indicates a loss of cell viability, this result can reflect a wide variety of processes unrelated to programmed cell death. Therefore, Annexin V binding/PI uptake measurements were used to measure specific cellular lineages in the human PBLs based on fluorescence activated cell scanning (FACS) analysis. PBLs were stained with anti-CD3, anti-Annexin V, and anti-PI antibodies immediately after isolation. After 12 or 24 hours in culture in the presence or absence of TNF-α, cells were analyzed by FACS for Annexin V/PI binding to defined lineages. Over the course of these studies several important differences emerged in the behavior of human PBLs compared to NOD splenocytes (Table 1). First, human PBL isolates contained a large amount of cell fragments and debris, both at baseline and after culture. This finding was not evident in the NOD splenocyte preparations. Comparing the responses of non-diabetic C57BL/6 murine splenocytes to non-diabetic humans PBLs, the viability of C57BL/6 splenocytes remained constant during a 24 hour period of culture in vitro, whereas a progressive increase in spontaneous cell death of human PBLs was regularly observed over this interval. This differential response of cell viability to culture in vitro may possibly reflect an intrinsic fragility of human PBLs and/or the greater rigors of cell isolation required for purification of human PBLs as compared with murine splenocytes. Many cells from the human PBL lineages most prone to apoptosis may die during cell isolation, as suggested by the contamination by cell fragments of the resulting isolated PBLs. Another difference between PBLs from human Type I diabetics and NOD splenocytes is that the evolution of TNF-α-induced apoptosis in NOD splenocytes occurs with a predictable time course that is much more rapid than that seen in PBLs from Type I diabetics. Thus, NOD splenocytes exhibited a clear increase in Annexin V binding after a 12-hour incubation with TNF-α. By 24 hours, there was little further increase in the fraction of cells binding Annexin V. However, nuclear binding of PI to NOD splenocytes had increased substantially reflecting a further progression of the apoptotic program. By contrast, cell death in response to TNF-α in human CD3 positive-PBLs, when evident, evolved much more slowly. For example, increased Annexin V binding was rarely seen after 12 hours and at 24 hours, increased Annexin V uptake, when present, was generally not accompanied by increased PI uptake. In addition, in vitro apoptosis in B-cell and macrophage lineages exhibited a much different time course than that of T-cells.

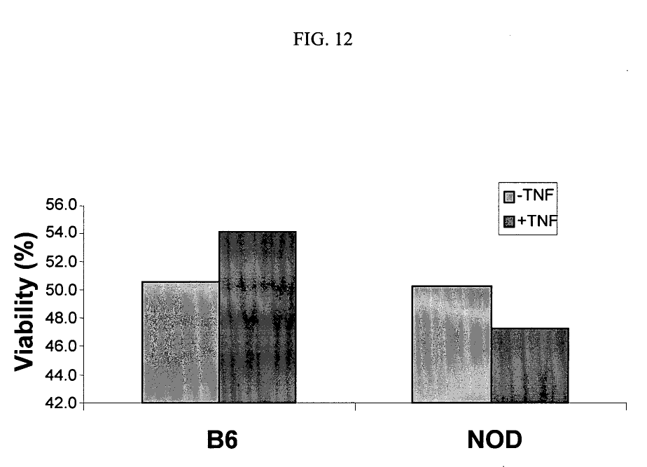

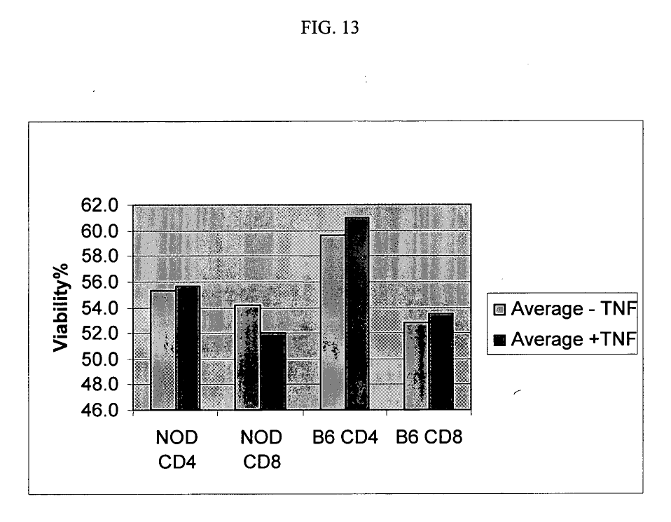

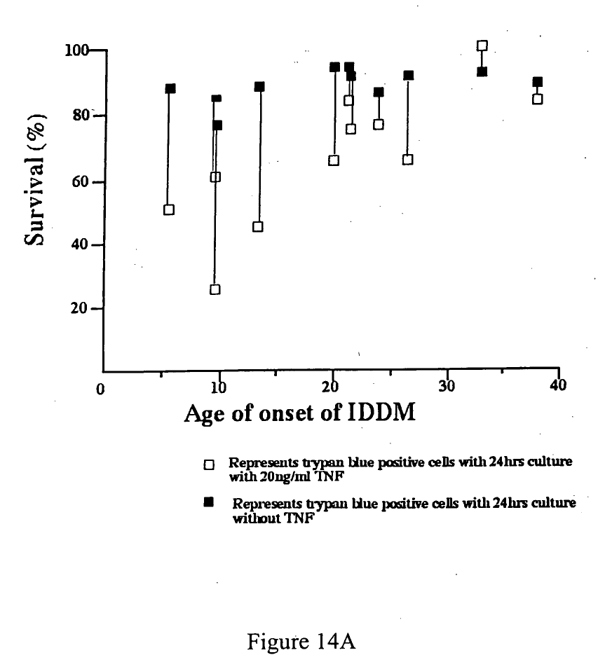

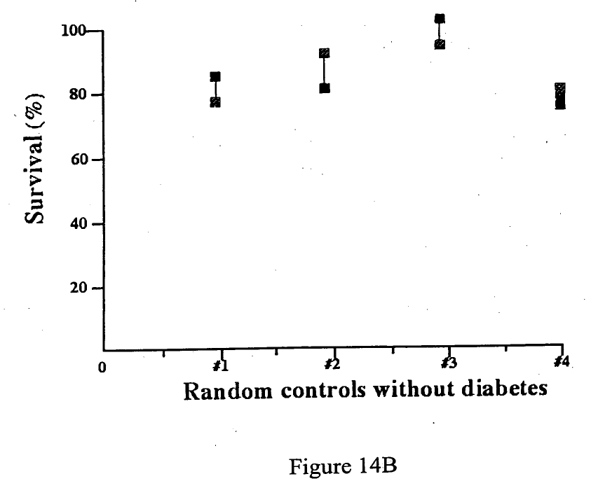

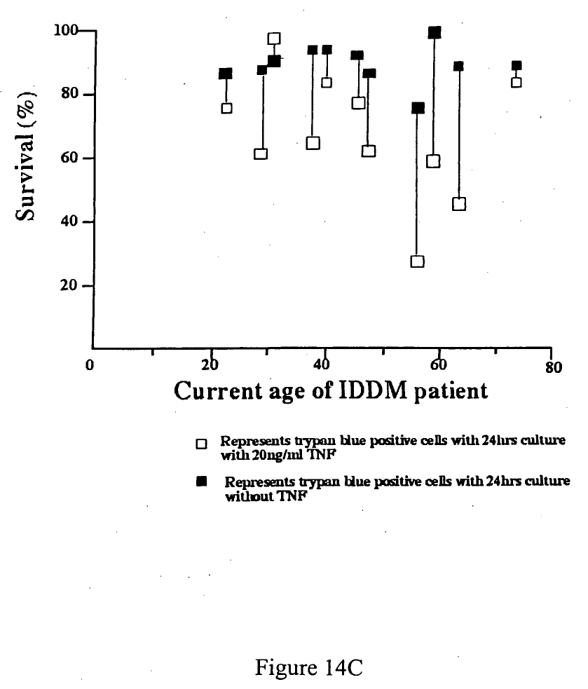

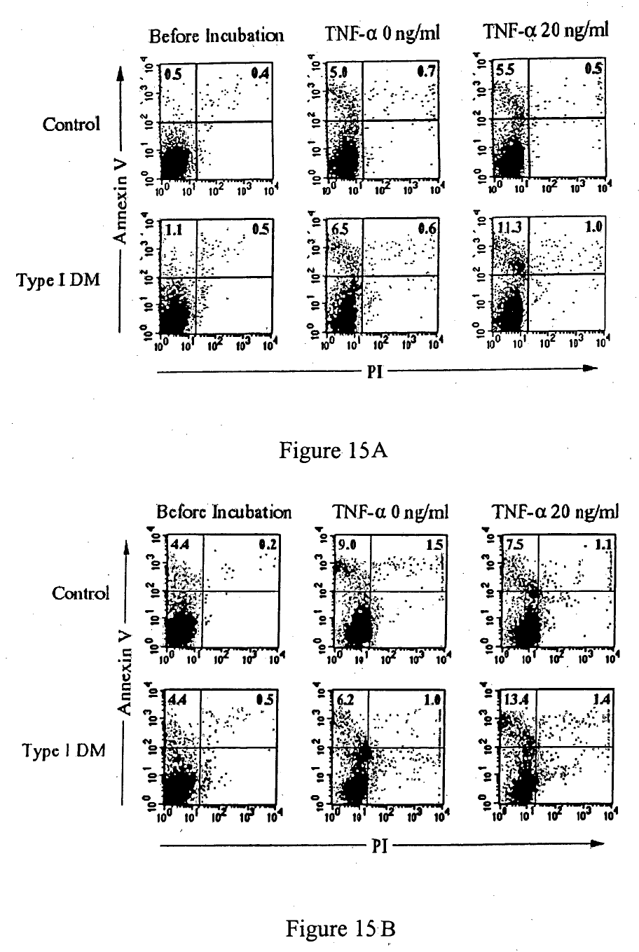

The results generated using CD3, CD19, and CD11b positive PBLs isolated from 33 Type I diabetics and from control subjects (see Table 1) support the following conclusions. First, one or more cellular lineages from a subset of Type I diabetics exhibit a significant increase in apoptosis on incubation with TNF-α in vitro. Two specific examples are shown in FIGS. 15A and 15B. In these studies, PBLs were processed in parallel from a Type I diabetic and an age/sex matched control non-diabetic subject. Immediately after isolation, the PBLs were incubated with Annexin V and propidium iodide and subjected to FACS analysis (left column of flow charts; “before incubation”). In FIG. 15A, the baseline staining by Annexin V was low (0.5 and 1.1%) whereas in FIG. 15B, a greater fraction (4.4%) of cells showed Annexin V binding at baseline. The cells were then incubated in the absence (center panels) or presence (right panels) of TNF-α (20 ng/mL). After 12 hours, these cells were stained with Annexin V and PI and analyzed by FACS. In all instances, the staining by Annexin V⁺PI⁺ increased significantly and selectively in PBLs from a diabetic patient.

Second, Annexin V staining increased substantially after culture, whether or not TNF-α was present. Notably, the increase in Annexin V staining occurring in the presence of TNF-α was markedly greater in the PBLs isolated from the Type I diabetic subjects (6.5% to 11.3%; 6.2% to 13.4%) than in the PBLs from the parallel non-diabetic control subjects (5.0 to 5.5%; 9.0 to 7.5%). This pattern is similar to the early stage of apoptosis seen in NOD splenocytes, except that uptake of PI had not occurred in the human PBLs at this time. When incubations were continued for 24 to 36 hours, the fraction of cells exhibiting increased uptake of PI increased substantially, although the variability in the levels of uptake increased. At extended times, the percentage of apoptotic cells may increase and then decrease. This trend occurs because cell death is followed by cell lysis, resulting in fewer apoptotic cells that remain intact and that can be measuring using this staining method.

The data obtained using human CD3⁺ T-cells differs in several respects from that obtained using NOD splenocytes. The increase in PI uptake is much slower in human T-cells than in the murine T-cells. For example, little PI uptake is evident after 24 hours in culture despite a doubling in the fraction of cells that bind Annexin V, both in the diabetic and control samples. Second, the increase in Annexin V binding to CD3⁺ PBLs observed after 24 hours in response to TNF-α (20 ng/1 nL), while greater to a statistically significant extent in the Type I diabetic PBLs than in the non-diabetic PBLs, is much smaller in magnitude than that seen for NOD splenocytes, and the variance is substantially greater.

TABLE 1
Comparison of overnight early T-cell death of patients compared to
random controls

| | | | | | |
|---|---|---|---|---|---|
| | | After culture | | After culture | |
| | Before culture | w/o TNF-α | | with THF-α | |
| n | (Mean % cell | (Mean % cell | | (Mean % cell | |
| (pairs) | death ± SE) | death ± SE) | p | death ± SE) | p |

Mouse

| | | | | | | |
|---|---|---|---|---|---|---|
| NOD | 5 | 9.9 ± 2.6 | 18.9 ± 3.14 | 0.002 | 32.9 ± 3.97 | 0.008 |
| C57BL/6 | 7 | 7.9 ± 2.1 | 10.7 ± 3.2 | 0.32 | 10.2 ± 2.5 | 0.44 |

Human

| | | | | | | |
|---|---|---|---|---|---|---|
| IDDM | 33 | 4.18 ± 0.48 | 7.84 ± 0.88 | 1 × 10⁻⁵ | 8.73 ± 0.96 | 0.025 |
| control | 3 | 3.90 ± 0.93 | 7.64 ± 0.76 | 5 × 10⁻⁶ | 8.02 ± 0.85 | 0.025 |

In practice, methods for the evaluation of human PBLs for apoptotic or necrotic events require more care than those used for murine splenocytes. Some methods of isolation of hPBLs took approximately 14 hours, compared to only about 45 minutes for preparation of murine cells for FACS analysis. Patient blood samples were collected in the patient care area, engendering substantial delays before processing can begin. The occurrence of spontaneous cell death in human PBLs after isolation increases with the time elapsed between obtaining a blood sample and isolation of PBLs. The standard isolation techniques for purification of human PBLs, when applied to C57BL/6 splenocytes, substantially increase the extent of spontaneous cell death during subsequent incubation with TNF-α. Different cellular subsets of human PBLs exhibit different time courses of cell death in response to a variety of perturbants. FACS analysis enables a restricted examination of time-dependent phenomena, because the analysis destroys the sample and thus prevents the sample from being re-examined at later timepoints.

Thus, despite the above evidence indicating the occurrence of a statistically significant increase in TNF-α induced T-cell death in vitro in PBLs from the human diabetics assessed by Trypan blue uptake and the subset of Type I individuals whose T-cells display an unmistakable apoptotic response to TNF-α in vitro as measured by Annexin V binding, the FACS assay of Annexin V binding/apoptosis in human PBLs as just described needed further modification to be more generally applicable as an assay of treatment efficacy. Therefore, new assay conditions were developed using automated technology for cell sorting and FACS analysis.

Optimized Assay

The methods described herein enable the effect of rapid testing of many candidate compounds on many lymphoid lineages and can be used to measure the effect of multiple treatment doses on PBLs from larger patient populations.

The methods also facilitate the detection of potentially pathogenic cell lineages, or lineages whose behavior in vitro parallels that of pathogenic lineages. These assays of cell death of PBLs also allow the identification of subsets of human patients (e.g., patients with Type I diabetes) responsive to additional pro-apoptotic ligands specific to pathologic cells.

The following section describes the streamlining of PBL preparation, optimization of assay conditions, and development of rapid, automated, high throughput methods for the in vitro assay of programmed cell death in PBLs. These methods can also be used to measure cell surface markers, such as CD45, CD62L, and/or CD95, as phenotypic assays.

Optimized Preparation of PBLs

The hypaque-ficoll density gradient method used above for the preparation of PBLs was replaced with a more rapid and less toxic methodology that can be largely automated. The use of a cell preparation tube, for example, the Vacutainer™ CPT™ Cell Preparation Tube from Becton-Dickinson, allows blood to be drawn directly into a buffered density gradient appropriate for cell separation, followed by immediate centrifugation. The advantages of this method over that previously described include less blood handling, manual pipetting, multiple centrifugations, and multiple washes. In addition, because PBL prepararation takes place over a shorter time frame, less cells are lost due to spontaneous cell death during the isolation process.

In addition to improved handling techniques, rapid red blood cell (RBC) lysis methods can be used for elimination of the majority of RBCs.

Commercially available systems include those from Ortho Orthimmune from Ortho, A Whittaker ACK lysis buffer from A. Whittaker, FACS lyse from BDL, and Optilyse from Immunotech. Other NH₄Cl lysis protocols (e.g., those based on Terstopper, et al., J. Immunol. Methods 123:103, 1989) can also be used. In a technique that is complementary to (or to be used in conjunction with) RBC lysing, RBC removal can be affected by the use of magnetic affinity purification techniques (e.g., by using an antiglycophorin A antibody attached to magnetic beads, BD Pharmigen, Catalog Nos. 32591A or 555569).

Optimized Assay Conditions for PBLs

Programmed cell death is a progressive multistep process and assays are available to monitor many of these steps. Early responses include phosphatidylserine externalization, which can be assayed by Annexin V binding or caspase cleavage, which can be assayed by cleavage specific antibodies. Intermediate steps include changes in mitochondrial membrane potential, release of mitochondrial polypeptides (e.g., Cytochrome C and AIP) into the cytoplasm, and caspase substrate cleavage (e.g., PARP). Late steps include DNA fragmentation (e.g., assayed by TUNEL assay) and loss-of-surface membrane integrity (e.g., assayed by uptake of Trypan blue, merocyanine 540, or Propidium Iodide dyes). Loss-of-membrane integrity is easily assayed but is a nonspecific manifestation of loss of viability. The Annexin V binding assay is a relatively reliable measure of programmed cell death in NOD splenocytes. A technical problem for the Annexin V assay is the apparent substantial binding of Annexin V to normal human B-cells and macrophages. Moreover, each major human mononuclear cell subset, i.e. T-cells, B-cells, and macrophage, differ in their response to TNF-α both in terms of the extent of cell death and its time course. A number of procedures have been recently employed in the optimization of fluorescence assays for evaluating the specific cytotoxicity of T cells (e.g., see Fischer et al., in J. Immunol. Methods 259:159-69, 2002) that can be used to further optimize assays for human PBLs that are described described herein.

The methods of the present invention can be used to measure the response of PBLs to a large variety of ligands from the TNF family, as well as to monoclonal antibodies to TNF family receptors. A large number of these molecules, e.g. FasL, TRAIL, and Apo3 are expressed in the lymphoid system and regulate cell proliferation and survival, often in opposing directions at different stages of lymphocyte development and during differing stages of cellular activation. The response of PBLs from non-diabetic control and Type I diabetic subjects to withdrawal of serum and growth factors, addition of pro-apoptotic agents that act through a variety of mechanisms (e.g., DNA damage and ROS generation), apoptosis accelerants, and cell cycle regulators can also be evaluated by the methods of the invention. Agents such as staurosporine or tamoxifen can initiate apoptosis directly or sub-apoptotic concentration of agents can be used to accelerate the evolution of apoptosis in response to a variety of receptor-initiated signals. The latter technique decreases the splay in the data that increases over time and is therefore of considerable practical value. As TNF-α has been reported to only kill cells in the G₂ phase of the cell cycle, synchronization of PBLs prior to TNF-α induced apoptosis may also be beneficial. Inhibitors of RNA or protein synthesis can also greatly enhance and accelerate the apoptotic response to TNF-α in many cell types. In immature T-cells, this effect is usually attributed to a suppression of the concomitant TNF-α induction of NF-κB. By contrast, TNF-α promotes apoptosis in memory T-cells without the addition of a RNA or protein synthesis inhibitors, possibly because of the maturation-induced expression of RIP.

If desired, the methods of the invention can include examination of specific subpopulations of autoimmune cells for susceptibility to cell death. In one example, T-lymphocytes can be visualized according to the self-antigens they recognize through the use of fluorescently-labeled MHC class I multimers, as reported by Sun et al. in Arthritis Res. 3:265-9, 2001. In another example, activated memory cells, e.g., those expressing CD25/IL2Rα, are examined. Yet another example involves the examination of B cells that overexpress B cell maturation protein (BCMA). These approaches for autoimmune cell identification can be directly applied to the flow cytometric techniques that are used in the methods of the invention.

Automation of Cell Death Assays for High-Throughput Cellular Screening

Flow cytometric analysis is useful as a reliable assay for the detection of heightened TNF-α sensitivity (e.g., TNF-alpha induced cell death) in autoimmune patients (e.g., human diabetics). This methodology and assessment of cell viability in a subpopulation of randomly selected diabetic subjects compared to matched controls indicates the existence of signaling defects in some diabetics. For human studies, one dose of TNF-α (20 ng/mL) was tested with a 12 or 24 hour co-incubation time.

Since the human diabetic population is heterogeneous, a method for reliably establishing sensitivity to candidate compounds using high-throughput platform cellular screening is used in the optimized assay. Typically, about 20 mL of blood is withdrawn from the patient followed by, if desired, processing of the leukocytes as described above. In the fully automated setup, automated workstations pipet blood from clinic blood tubes directly to 96 or 384 wells for RBC lysis and/or removal, followed by programmable washes, dilutions, and suctions to add the desired ligands and fluorescent probes to quantify cell viability. Such a setup optimizes speed and assay standardization for diagnosing autoimmune disease or monitoring autoimmune therapy that includes the isolation or depopulation of leukocyte cell populations or subsets of leukocyte populations.

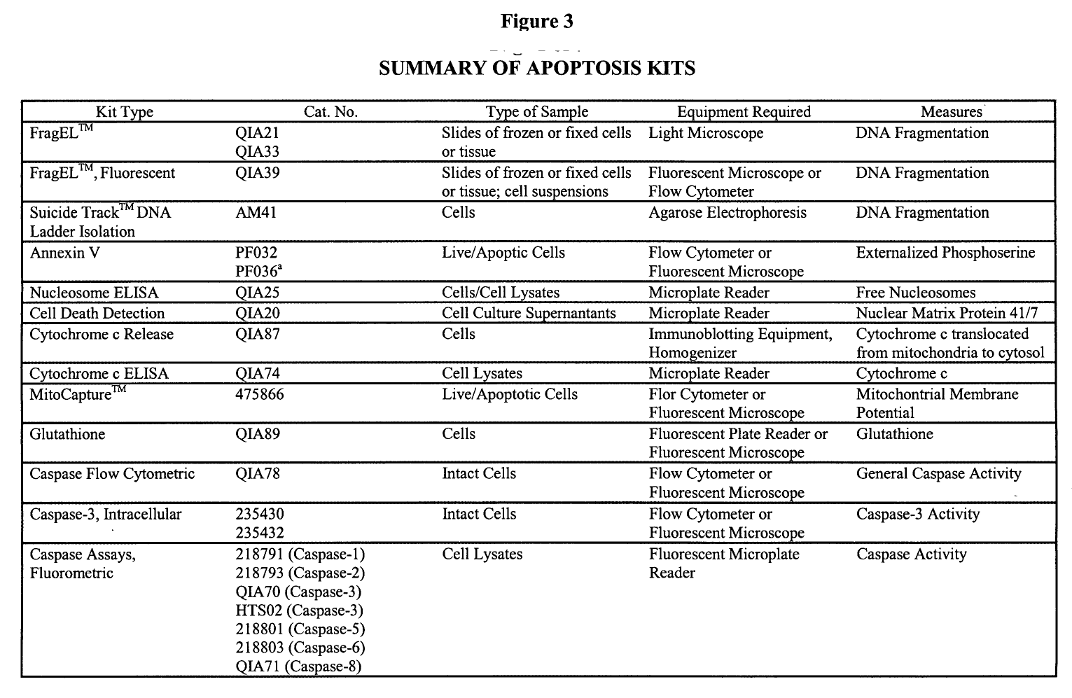

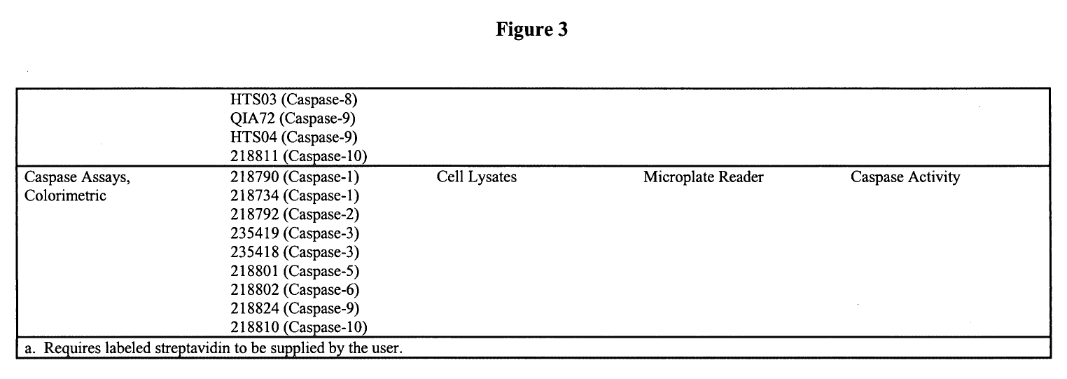

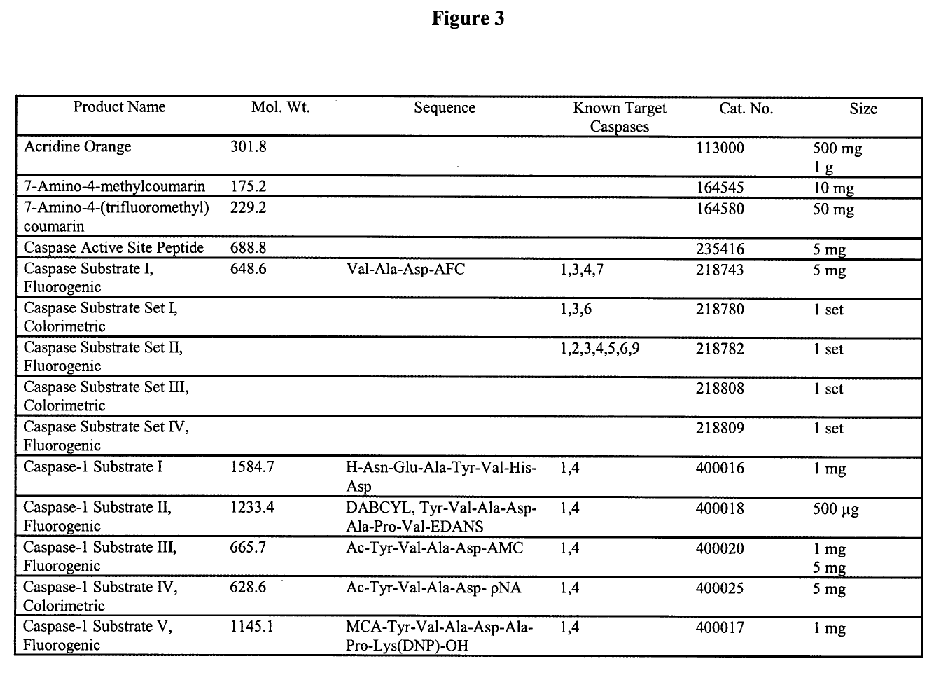

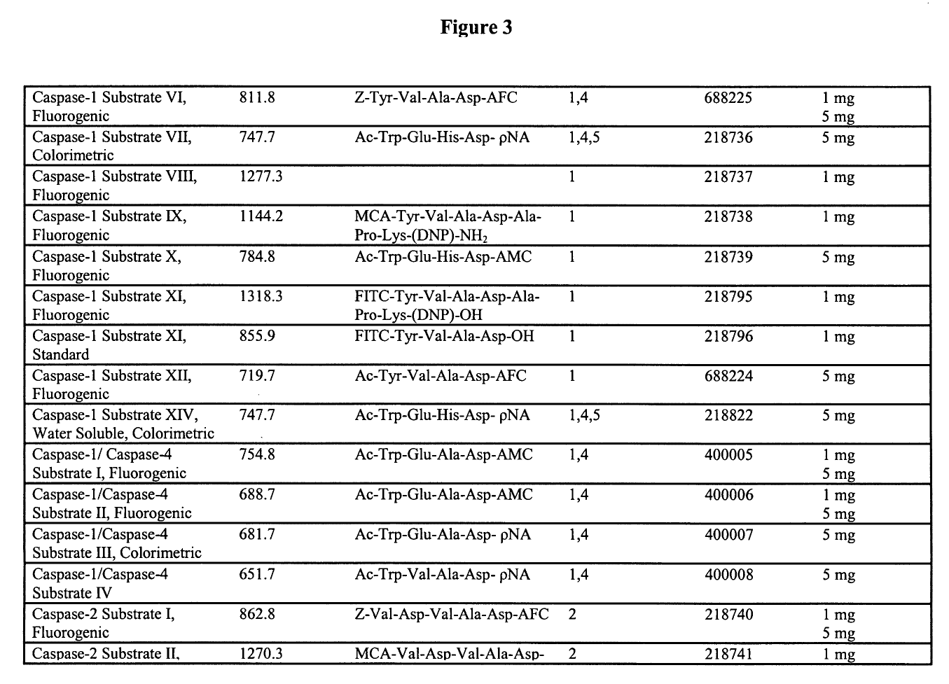

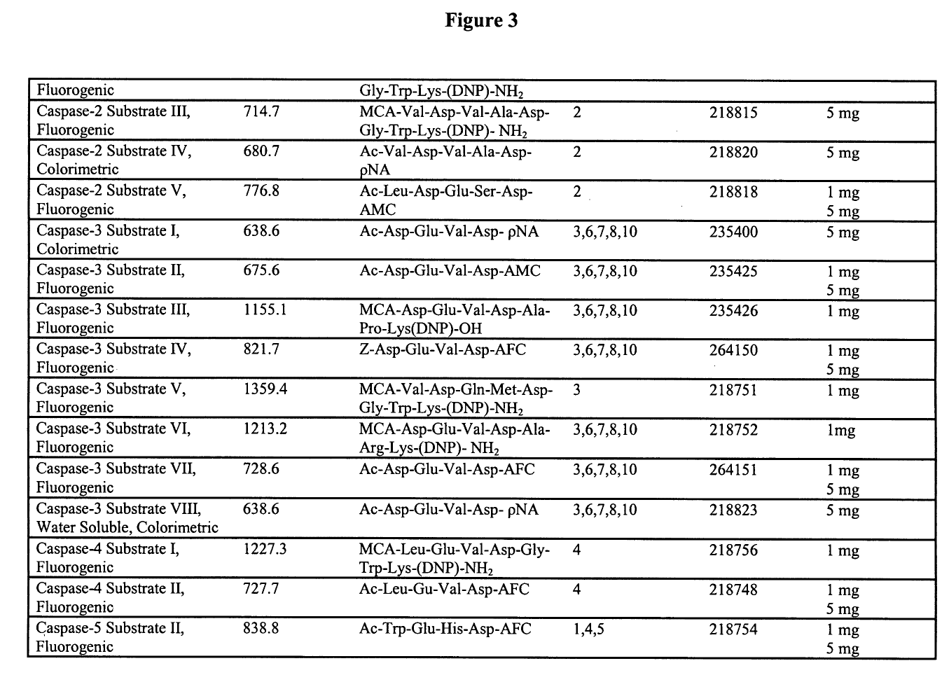

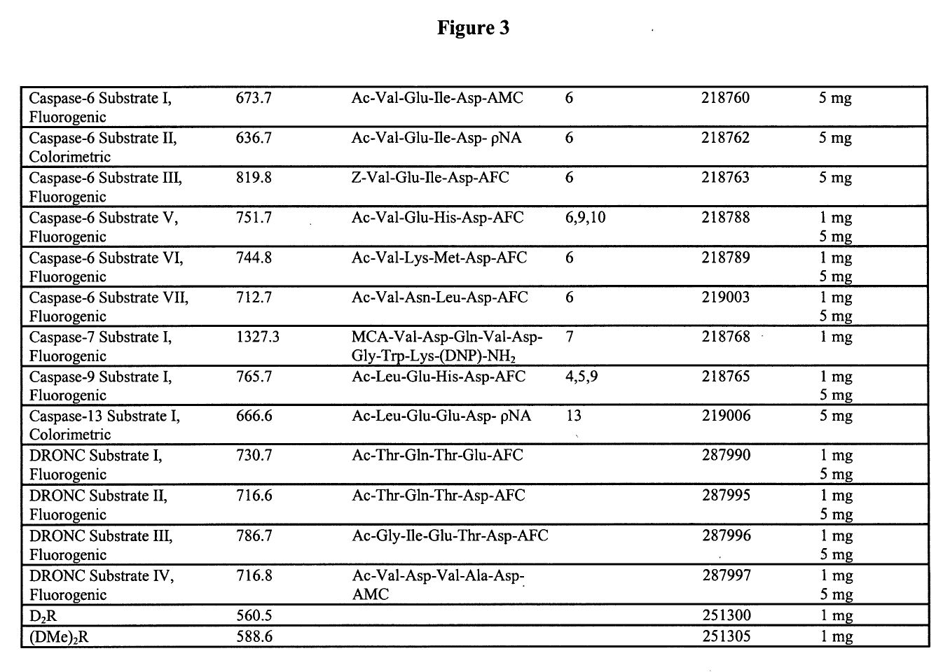

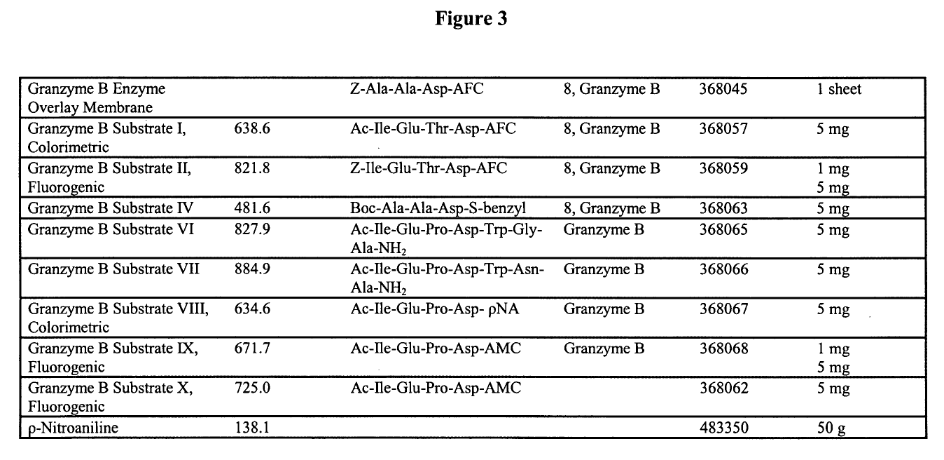

Once the blood has been processed, compounds and apoptosis kits (see FIG. 3) are applied to the blood sample. Typically, high-throughput platform cellular screening involves 96 or 394 well plates containing 500 to 1000 cells per well that are read by a multi-parameter fluorescent reading machine that analyzes the cells without their removal from the plate. The viability or death of specific cell populations is examined at 10-20 different concentrations of leukocyte-killing compound (e.g. TNF-α), at 5-10 analysis times points for the same cells, with 2-5 markers of apoptosis or cell death or ligands as a measure of cell viability or death. A variety of kits for measuring apoptosis are available and are known to those skilled in the art (also see FIG. 3). High-throughput cellular screening also enables the rapid, quantitative analysis of small amounts of human blood to measure the phenotype penetrance of heightened apoptosis. Once the penetrance of the apoptotic phenotype is detected, the same assay can be used in to measure the effect of a candidate compound in vivo.

In addition to diagnosing and monitoring autoimmune disease, the automated assays are also useful for screening compounds (e.g., cytokines, peptides, and antibodies) for the desired effects of selective death of specific subpopulations of autoreactive cells. Combinations of compounds may also be tested. For example, many autoimmune diseases include two subpopulations of autoreactive cells in different stages of activation and with different susceptibilities to killing agents.

As an alternative to the Cellomics scanner, a Universal Imaging Corporation scanner can be used. Other commercially available scanners, pipetters, etc. are also suitable for automation of the methods of the invention and are known to those skilled in the art.

Screening Assay for hPBLs

Peripheral blood lymphocytes (PBLs) represent a particular challenge for high-throughput plate-based assays. These cells often do not stick to surfaces, and cells that die often lose adhesion. These factors mean that certain measures such as those described herein should be taken to have these cells stick to the plate surface, whether they are living or dead. Desirably, the assay kit involves as few washes as possible.

Matrix adhesion is one way to adhere PBLs to a surface. Unfortunately, cells the underlying extra cellular matrix can increase cellular susceptibility to death following DNA damage or cell culture. Furthermore, a vigorous wash may also cause loss of adhesion from the ECM, which may trigger apoptosis in even normal cells, as described by Greenwood in Nature Reviews Cancer 2:323 (2002). A recent report by Lewis, et al., in Proc. Nat. Acad. Sci. USA 99:3627-32 (2002) shows that ligation of cells to integrin surfaces that mediate the attachment of cells to the ECM increased the level of apoptosis in a tumor cells line. An assay to measure cell death desirably minimizes or avoids the following: plate coating with cell adhesion, many washes, matrix adhesion, cross-linking of the cells by integrins or antibodies that may change the death process, and detachment of cells, from either washes or changes in the surface.

Thus, to measure apoptosis or other forms of cell death, the following technique was developed. Instead of adhesion procedures, a full semi-solid suspension is used to immobilize the PBLs prior to the initiation of the assay. For example, cells may be mixed with alginate or agarose, and then the mixture placed in the assay well. The alginate is then solidified with Ca₂PO₄, and the assay begun, using an apoptosis kit and an automated scanner in a flow cytometric procedure.

The use of alginate has the advantage of not requiring phenol for imaging. Alginate also allows cells to maintain their natural round shape, which is a major physiological advantage compared to flattened cells on a matrix support. Additionally, the alginate maintains the cells in a fixed location even after death, allowing even late stages of death to be monitored. Furthermore, multiple washes can be performed without loss of the dead cells or a change in the position of the cells.

The automated assay as described can be used for the examination of leukocyte cell death, regardless of the cell death pathway that was activated, or the examination of one or more specific cell death pathways. For example, necrosis may be monitored, e.g., by caspase independence, swelling of cells and organelles, cellular disintegration, and/or release of cytoplasm materials.

Following is an optimized protocol for the automated analysis of cell sensitivity to compounds that induce cell death via apoptosis or necrosis.

I. Collect subject/control blood sample in anticoagulant tube:

II. Remove red blood cells and collect lymphocyte subsets:

1. Transfer 2 mL of whole blood to a 15 mL tube

2. Add 50 μL reagent subset CD-marker to each whole blood sample.

3. Mix/vortex

4. Incubate 15 min. at 4-8 degrees C.

5. Add 10-12 mL sample reagent media.

6. Gently mix

7. Spin (445 g) for 10 min. at room temperature

8. Aliquot (remove and discard) supernatant

9. Mix

10. Add 1-3 mL sample reagent media.

11. Gently mix.

12. Place on AutoMACS cell sorter for CD-subset separation (magnetic bead separation method or direct RBC lysis)

13. Label/Prepare 15 mL collection tube

14. Add 200 μL 100% FCS to a 15 mL subset collection tube.

15. Place collection tube on AutoMACS/magnetic cell separation machine.

16. Collect 2 mL magnetically purified and separated lymphocyte sample (the AutoMACS sample) into collection tube.

III. Cell Counting:

1. Remove a 15 μL-50 μL sample aliquot from magnetically-separated lymphocyte sample.

2. Add 15 μL-50 μL counting dye to sample aliquot.

3. Mix

4. Add sample aliquot to a cell counting instrument.

5. Adjust cell volume of the AutoMACS sample so that the cell concentration will contain 25 k cells in 100 μL.

a. Centrifuge sample lightly

b. Remove supernatant

c. Re-suspend sample

IV. 96-Well Plate:

1. Pipette 100 μL cell suspension (25,000 cells) into each 96-well plate well, ×3 for triplicates

2. Add 5 μL of reagent (e.g., TNF-α, another induced or cell death, or mixtures containing up to 10 different chemical reagent combinations for the induction of cell death), to each well to be treated, ×3 for triplicates.

3. Add 5 μL of media to the control cells' wells, x3 for triplicates

V. Incubation:

1. Place 96-well plate into incubator and incubate at 35° C. for 2, 4, 6, 8, 10, 12, 14, 16, 18, 20 or 22 Hrs.

VI. Plate Analyzer/Reader (Automation Sequence for Liquid Handling)

1. Remove sample plate from incubator

2. Remove identical control sample plate from incubator

3. Put 96-well sample plate on automated cell reader for total cell counts for each well.

4. Put 96-well control plate on automated cell reader for total cell counts for each well

5. For flow cytometry (analysis), remove 90 μL of re-suspended sample material from each sample well

6. Transfer sample material to (10 mL) flow cytometry test tube

7. Add 90 μl of cell stain solution to each sample flow test tube above to stain cells for identification

8. Incubate 10-15 minutes at room temperature in the dark

9. Add 320 μL reagent mix

10. Place incubated sample tube on flow cytometer (analyzer)

11. Acquire timed and/or absolute count/volume data

12. Remove sample tube from flow cytometer

13. Add next incubated sample tube to analyzer

14. (If doing plates, return plate to incubator)

15. Repeat measurement at specified time interval(s).

For the protocol outlined above, it is possible to enrich for autoreactive cells first by taking the leukocyte fraction, after removal of RBCs, and enriching it further by fractionating it into autoreactive subsets with MHC class I and self peptide complexes. These complexes could be linked together as tetramers (see U.S. Pat. No. 6,248,564 or Xu et al., J. Immunol. Methods 268:21-8, 2002) or plated monomers onto plastic plates. In one example, for the nearly 94% of Caucasian humans that are HLA A2/HLA B7 positive, selective removal of these subtypes of cells would yield a well containing a concentrated amount of pathogenic cells. The additional advantage of tetramers is that micro-wells with cell numbers of only 10 cells/per well could be used and studied by scanning/imaging methods of cell death. First tetramers could be plated onto wells and secondly, the cells binding could then adhere to the surface for serial study with imaging.

Non-T cell population of pathogenic cells has also been identified by their increased tendencies to under go spontaneous death (e.g., those that are CD180 deficient). The automated format outlined above can also include non-T cell fractions that are analyzed separately. These fractions can be obtained by the affinity-enrichment techniques (e.g., magnetic cell sorting) already described or others that are known in the art.

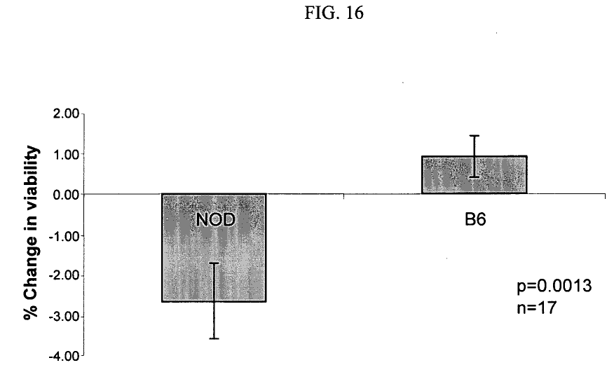

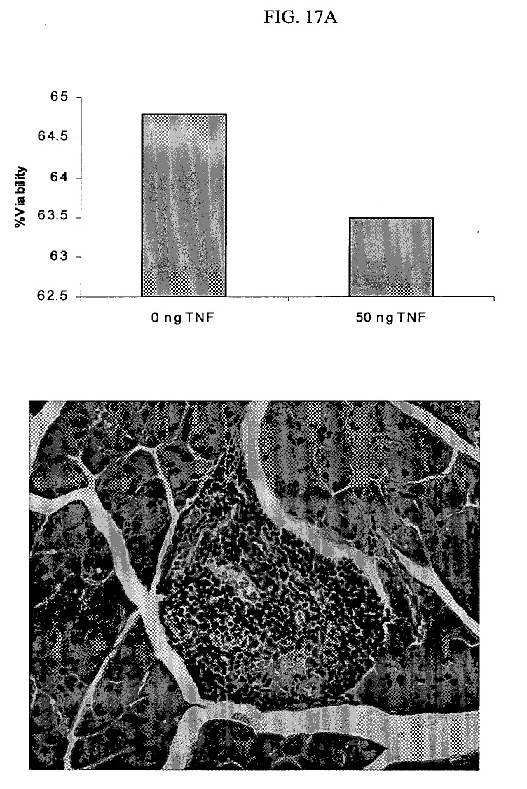

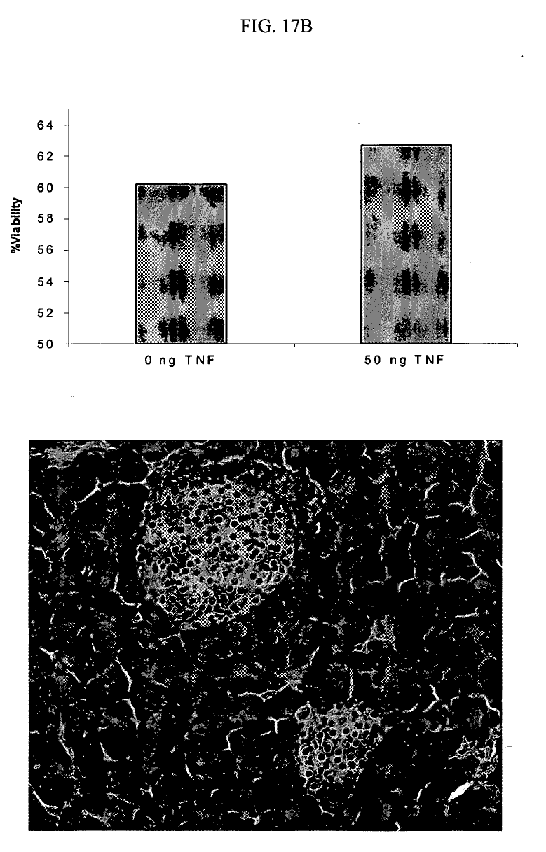

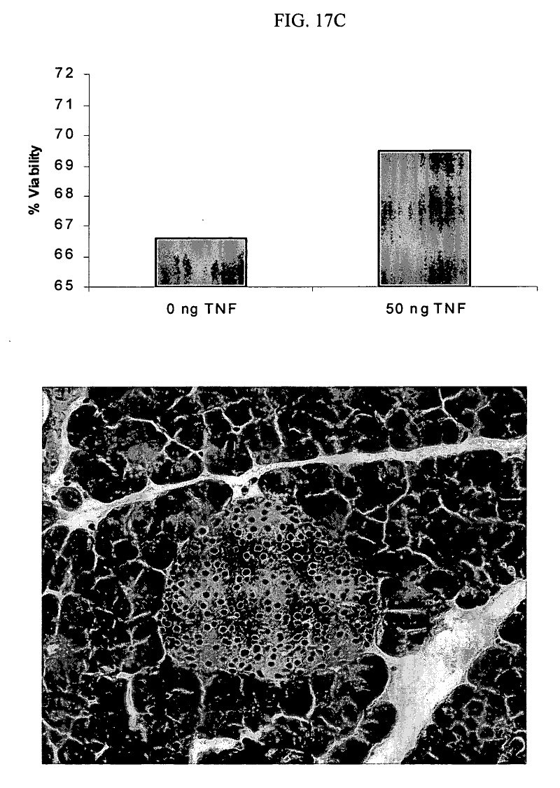

The sensitivity of T cell populations from NOD mice were re-examined using the automated assay described above. A statistical analysis of splenocyte viability in NOD and B6 mice (FIG. 16, n=17 for each) treated with TNF-α indicated that the diabetic mice had a −2.7+/−0.9 decrease in viability while the control B6 mice had a 1.0+/−0.5 increase in viability. The cells of individual mice from these cohorts were then examined for TNF-α sensitivity in cell culture and these results were correlated with a histological examination (see FIGS. 17A, 17B, and 17C, staining was done by H&E on parafin embedded pancreatic tissue, with lymphoyctes staining as black dots which are easily distinguished from the exocrine and endocrine portions of the pancreas). FIG. 17A shows splenocytes from one NOD mouse treated with TNF-α in a cell culture experiment. The treated splenocytes showed decreased viability vs. those that were not treated with TNF-α (63.5% viability vs. 64.7% viability, respectively). The histological results from this mouse, also shown in FIG. 17A, are typical of a diseased animal and confirm the cell culture result. The splenocyte cell viability of another NOD mouse is graphed in FIG. 17B, In the cell culture experiment, the splenocytes showed increased viability vs. those that were not treated with TNF-α (63.0% viability vs. 60.2% viability, respectively). The histological results from this mouse are typical of an animal that is not suffering from diabetes, again confirming the cell culture result. The splenocyte cell viability of a control B6 mouse are graphed in FIG. 17C, showing increase viability of splenocytes treated with TNF-α vs. those that were not treated with TNF-α (69.7% viability vs. 66.6% viability, respectively), with the histology typical for a normal animal, again confirming the cell culture result.

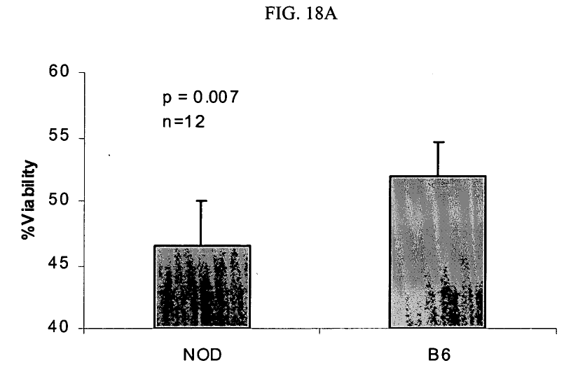

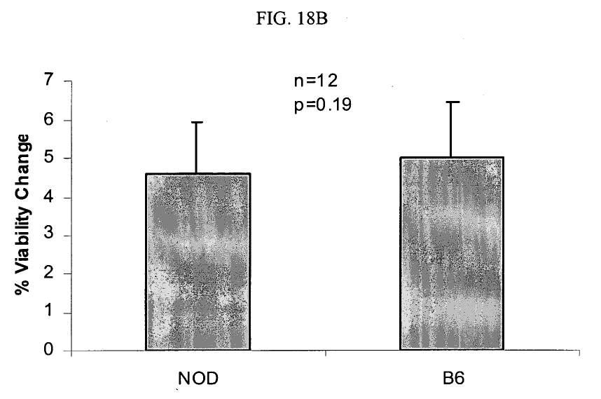

Non-T cells from NOD and B6 mice were also examined for sensitivity to TNF-α-induced cell death. Graphed in FIG. 18A are the viability results of untreated non-T cells (n=12 mice from each cohort). The results obtained show tissue culture death at an accelerated rate for non-T cells in NOD mice (48.0% viability vs. 53.7% viability for NOD vs. B6 mice, respectively). However, a shown in FIG. 18B when the untreated cells are compared with those treated with TNF-α, there is little TNF-α sensitivity observed. (4.6% viability change vs. 5.0% viability change for NOD vs. B6 mice, respectively).

The automated method described herein has also been used for the assay of cell viability in a human patient. Blood samples were obtained from a patient with Type I diabetes and from a non-diabetic control subject. During the assay procedure, a CD3⁺ T cell subset was isolated from each (Step 11.12 from the protocol). The cells were then contacted with TNF-α and cell viability was measured and compared to that found in control samples which were not contacted with TNF-α. The results, presented in Table 2, show a relative decrease in viability for those CD3⁺ cells exposed to TNF-α in the diabetic patient, demonstrating the increased susceptibility of autoimmune cells to reagents that induce cell death via apoptosis and the use of this property for the diagnosis of autoimmune disease.

TABLE 2
Comparison of CD3⁺ cell viability in a patient with Type I diabetes to a
subject without Type I diabetes

| | | Type I diabetic | | Control | |
|---|---|---|---|---|---|
| Viability (%) | TNF-α | − | + | − | + |
| | | 95 | 93 | 95 | 96 |
| Death (%) | TNF-α | − | + | − | + |
| | | 5 | 7 | 5 | 4 |
| Total Cell Count | TNF-α | − | + | − | + |
| | | 1900 | 1900 | 2000 | 2200 |

### EXAMPLE 3

### Assay for the Diagnosis of Autoimmune Disease by the Examination of B Cell Populations by Mass Spectrometry

In addition to the use of FACS analysis, other analytical techniques can be employed by the analysis of cell populations. CD180 (RP105) is a toll-like receptor (TLR) that is critical for the response of B cells to bacterial lipopolysaccharide (LPS). It has been shown that B cells lacking the CD180 molecule, which proved to be highly activated B cells, are increased in the peripheral blood of patients with systemic lupus erythematosus (Koarada et al, Arthritis Rheum. 42:2593-600, 1999).

In the present invention, whole splenocytes from NOD and B6 mice (normal control) greater than 12 weeks of age were analyzed by mass spectrometry for the presence of CD180. The lymphoid cells were separated into non-T-cell and T-cell populations, both of which were similarly analyzed by mass spectrometry. It was found that CD180 protein was detected in the non-T cell fraction of the control mice but not in the NOD mice. As expected, T cells from both NOD mice and B6 mice did not express CD180, as this protein is believed to be restricted to B cells.

It was then found that, in the NOD mouse, BCG administration kills the subpopulation of B cells that are CD180-deficient. In one experiment, NOD and B6 mice were subjected to BCG treatment via one subcutaneous injection in the footpad. Two days after BCG treatment, the splenocytes were removed and examined for CD180 antigen, at which time both B6 and NOD mice showed equivalent amounts of CD180 antigen in the non-T cell populations. These results were confirmed with analysis by Western gels. These results show that BCG treatment decreased or removed the population of autoimmune cells that are CD180-deficient.

Pharmaceutical Therapeutics

The methods of the invention provide a simple means for identifying compounds (e.g., peptides, small molecule inhibitors, and mimetics) capable of treating or preventing autoimmune disease. Accordingly, chemical entities discovered to have medicinal value using the methods described herein are useful as drugs or as information for structural modification of existing compounds for treating autoimmune disease, e.g., by rational drug design. For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections, which provide continuous, sustained levels of the drug in the patient. Treatment of human patients or other animals is carried out using a therapeutically effective amount of an agent in a physiologically-acceptable carrier. Suitable carriers and their formulation are described, for example, in Remington's Pharmaceutical Sciences by E. W. Martin. The amount of the agent to be administered varies depending upon the manner of administration, the age and body weight of the patient, and with the type of disease and extensiveness of the disease. Generally, amounts will be in the range of those used for other agents used in the treatment of other autoimmune diseases, although in certain instances lower amounts will be needed because of the increased specificity of the compound.

From the foregoing description, it will be apparent that variations and modifications may be made to the invention described herein to adopt it to various usages and conditions. Such embodiments are also within the scope of the following claims.

Information relevant to the present methods is disclosed in U.S. patent application Ser. No. 09/521,064, filed Mar. 8, 2000, Ser. No. 09/768,769, filed January 23, 3001, and 60/392,687, filed Jun. 27, 2002, and PCT publication WO00/53209, published Sep. 14, 2000, which are incorporated by reference. All publications and patent applications mentioned in this specification are herein incorporated by reference to the same extent as if each independent publication or patent application was specifically and individually indicated to be incorporated by reference.

What is claimed is:

1. A method for screening a test compound for the potential to prevent, stabilize, or treat an autoimmune disease comprising the steps of:
a) contacting a first blood sample from a first mammal that has, or is at risk for developing, an autoimmune disease with said test compound;
b) contacting a second blood sample from a second mammal that does not have, or is not predisposed to developing, an autoimmune disease with said test compound, wherein said first and second mammals are of the same species and said first and second blood samples are contacted with said test compound in the same manner; and
c) measuring leukocyte viability in said first and second samples, wherein said compound is determined to have said potential if said leukocyte viability in said first sample is decreased relative to said leukocyte viability in said second sample.

2. The method of claim 1, wherein said sample of step a) comprises leukocytes that overexpress a receptor for the group consisting of FasL, TNF-alpha, IL-1beta, IL-6, IL-12, and IFN-gamma.

3. The method of claim 1, wherein said sample of step a) comprises leukocytes that are deficient in the expression of CD180.

4. The method of claim 1, wherein said first blood sample comprises autoimmune splenocytes, autoimmune T lymphocytes, autoimmune B lymphocytes, or autoimmune cells of bone marrow origin.

5. The method of claim 1, wherein an apoptotic event is used to measure said viability.

6. A method for screening a test compound for the potential to prevent, stabilize, or treat an autoimmune disease comprising the steps of:
a) partitioning a first blood sample from a first mammal that has, or is at risk for developing, an autoimmune disease into a first fraction which is contacted with an inhibitor of apoptosis and a second fraction which is not contacted with an inhibitor of apoptosis;
b) contacting said first and second fractions with an inhibitor of apoptosis;
c) measuring the ratio of leukocyte viability in said first fraction of step b) to the leukocyte viability in said second fraction of step b);
d) partitioning a second blood sample from a second mammal of the same species as said first mammal, wherein said second mammal does not have, or is not at risk for developing, an autoimmune disease, into a third fraction which is contacted with an inhibitor of apoptosis and a fourth fraction which is not contacted with an inhibitor of apoptosis;
e) contacting said third and fourth fractions with said inhibitor of apoptosis in the same manner that said first and second fractions are contacted with said inhibitor; and
f) measuring the ratio of leukocyte viability in said third fraction of step e) to the leukocyte viability in said fourth fraction of step e),
wherein said compound is determined to have said potential if said ratio of step c) is greater than the ratio of step f).

7. The method of claim 1, wherein a necrotic event is used to measure said cell viability.

8. A method for screening a test compound for the potential to prevent, stabilize, or treat an autoimmune disease comprising the steps of:
a) partitioning a first blood sample from a first mammal that has, or is at risk for developing, an autoimmune disease into a first fraction which is contacted with an inhibitor of necrosis and a second fraction which is not contacted with an inhibitor of necrosis;
b) contacting said first and second fractions with an inhibitor of necrosis;
c) measuring the ratio of leukocyte viability in said first fraction of step b) to the leukocyte viability in said second fraction of step b);
d) partitioning a second blood sample from a second mammal of the same species as said first mammal, wherein said second mammal does not have, or is not at risk for developing, an autoimmune disease into a third fraction which is contacted with an inhibitor of necrosis and a fourth fraction which is not contacted with an inhibitor of necrosis;
e) contacting said third and fourth fractions with said inhibitor of necrosis in the same manner that said first and second fractions are contacted with said inhibitor; and
f) measuring the ratio of leukocyte viability in said third fraction of step e) to the leukocyte viability in said fourth fraction of step e),
wherein said compound is determined to have said potential if said ratio of step c) is greater than the ratio of step f).

9. A method for screening a test compound for the potential to prevent, stabilize, or treat an autoimmune disease comprising the steps of:
a) contacting a population of autoimmune cells from a mammal that has, or is at risk for developing, an autoimmune disease with said test compound;
b) contacting a second blood element from said mammal with said test compound, wherein said autoimmune cells and said second blood element are contacted with said test compound in the same manner; and
c) measuring the viability of said autoimmune cells, wherein said test compound is determined to have said potential if the viability of said autoimmune cell decreases relative to said second blood element.

10. The method of claim 9, wherein said second blood element is a non-autoimmune leukocyte.

11. The method of claim 9, further comprising the steps of:
i) prior to step a), partitioning said population of autoimmune cells of into a first fraction which is contacted with said compound and a second fraction which is not contacted with said compound; and
ii) after step b), measuring the ratio of viable autoimmune cells of said first fraction to said second blood element and measuring the ratio of said second fraction to said second blood element,
wherein said compound is determined to have said potential if said ratio of said first fraction to said second blood element is less than said ratio of said second fraction to said second blood element.

12. A method for diagnosing an autoimmune disease, or a predisposition to said disease, in a mammal comprising the steps of:
a) obtaining a first blood sample from a first mammal;
b) obtaining a second blood sample from a second mammal of the same species as said first mammal, wherein said second mammal does not have, or is not at risk for developing, said autoimmune disease;
c) contacting said first blood sample and said second blood sample with a compound that preferentially decreases the viability of leukocytes, wherein both of said first and second samples are contacted with said compound in the same manner; and
d) measuring the viability of leukocytes in said first blood sample and in said second blood sample, wherein a decrease in the leukocyte viability in said first sample relative to the leukocyte viability in said second sample indicates that said first mammal has, or is predisposed to developing, said autoimmune disease.

13. The method of claim 12, wherein said autoimmune disease is Alopecia, Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiffman Syndrome, Takayasu Arteritis, Temporal Arteritis/Giant Cell Arteritis, Ulcerative Colitis, Uveitis, Vasculitis, Vitiligo, Wegener's Granulomatosis, or myasthenia gravis.

14. The method of claim 13, wherein said autoimmune disease is Insulin-dependent Diabetes.

15. The method of claim 12, wherein said leukocytes overexpress a receptor for the group of chemokines consisting of FasL, TNF-alpha, IL-1beta, IL-6, IL-12, and IFN-gamma.

16. The method of claim 12, wherein said leukocytes overexpress B cell maturation protein (BCMA).

17. The method of claim 12, wherein said leukocytes are deficient in the expression of CD180.

18. The method of claim 12, wherein said mammal is a human.

19. The method of claim 12, wherein said compound is TNF-alpha.

20. The method of claim 12, wherein said compound is a TNF-alpha receptor agonist.

21. The method of claim 20, wherein said compound is a humanized or human monoclonal antibody.

22. The method of claim 12, wherein said compound binds to a Toll-like receptor on a B cell.

23. The method of claim 22, wherein said compound is BCG.

24. The method of claim 22, wherein said cell is deficient in the expression of CD180.

25. A method for diagnosing an autoimmune disease, or a predisposition to said disease, in a mammal comprising the steps of:
a) contacting autoimmune cells from a mammal that has an autoimmune disease, or is at risk for developing an autoimmune disease, with said test compound;
b) contacting a second blood element from said mammal with said test compound, wherein said autoimmune cells and said second blood element are contacted with said compound in the same manner; and
c) measuring the viability of said autoimmune cells, wherein said mammal has, or is at risk for developing, said autoimmune disease if the viability of said autoimmune cells decreases relative to said second blood element.

26. The method of claim 25, wherein said autoimmune disease is Alopecia, Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiffman Syndrome, Takayasu Arteritis, Temporal Arteritis/Giant Cell Arteritis, Ulcerative Colitis, Uveitis, Vasculitis, Vitiligo, Wegener's Granulomatosis, or myasthenia gravis.

27. The method of claim 26, wherein said autoimmune disease is Insulin-dependent Diabetes.

28. The method of claim 25, wherein said second blood element is a non-autoimmune leukocyte.

29. The method of claim 25, further comprising the following steps:
i) prior to step a), obtaining a first blood sample from said mammal and measuring the ratio of viable autoimmune cells to said second blood element in said first blood sample;
ii) during step a), administering said compound to said mammal;
iii) after step a), obtaining a second blood sample from said mammal and measuring the ratio of viable autoimmune cells to said second blood element in said second blood sample,
wherein said mammal has, or is predisposed to having, said autoimmune disease if the ratio of step iii) is less than the ratio of step i).

30. The method of claim 25, wherein said mammal is a human.

31. The method of claim 25, wherein said compound is TNF-alpha.

32. The method of claim 25, wherein said compound is a TNF-alpha receptor agonist.

33. The method of claim 32, wherein said compound is a humanized or human monoclonal antibody.

34. The method of claim 25, wherein said compound binds to a Toll-like receptor on a B cell.

35. The method of claim 34, wherein said compound is BCG.

36. The method of claim 34, wherein said cell is deficient in the expression of CD180.

37. A method for the stratification of a human patient into a therapeutic subgroup for an autoimmune disease comprising the steps of:
a) contacting a blood sample from said patient with a compound that preferentially decreases the viability of leukocytes;
b) measuring the viability of said leukocytes; and
c) placing said patient into a therapeutic subgroup based on the amount of decrease of said viability.

38. The method of claim 37, wherein said leukocytes overexpress a receptor for the group of chemokines consisting of FasL, TNF-alpha, IL-1beta, IL-6, IL-12, and IFN-gamma.

39. The method of claim 37, wherein said leukocytes overexpress B cell maturation protein (BCMA).

40. The method of claim 37, wherein said leukocytes are deficient in the expression of CD180.

41. The method of claim 37, wherein said viability is measured relative to the cell viability of leukocytes in a second blood sample obtained from a human that does not have, or is not predisposed to having, said autoimmune disease, wherein said second blood sample and said blood sample of step (a) are contacted with said compound in the same manner and said patient is placed into said subgroup based on the relative decrease of said viability.

42. The method of claim 37, wherein said viability is a measure of leukocyte viability in said sample relative to a second blood element in said sample, wherein said second blood element and said blood sample of step (a) are contacted with said compound in the same manner and said patient placed into said subgroup based on the relative decrease of said viability.

43. The method of claim 42, wherein said second blood element is a non-autoimmune leukocyte.

44. The method of claim 42, wherein said compound is administered to said patient.

45. The method of claim 42, further comprising the following steps:
i) prior to step a), obtaining a first blood sample from said mammal and measuring the ratio of viable autoimmune cells to said second blood element in said first blood sample;
ii) during step a), administering said compound to said mammal;
iii) after step a), obtaining a second blood sample from said mammal and measuring the ratio of viable autoimmune cells to said second blood element in said second blood sample,
wherein the decrease in the ratio of step iii) to the ratio of step i) determines the severity or course of said disease.

46. The method of claim 37, wherein said autoimmune disease is Alopecia, Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, Insulin dependent Diabetes, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiffman Syndrome, Takayasu Arteritis, Temporal Arteritis/Giant Cell Arteritis, Ulcerative Colitis, Uveitis, Vasculitis, Vitiligo, Wegener's Granulomatosis, or myasthenia gravis.

47. The method of claim 46, wherein said autoimmune disease is Insulin-dependent Diabetes.

48. A method for monitoring a therapy for a human that has an autoimmune disease, or is at risk for developing said disease, comprising the steps of:
i) obtaining a first blood sample from said patient and contacting said first blood sample with a compound that preferentially decreases the viability of leukocytes;
ii) measuring the viability of leukocytes in said first blood sample;
iii) obtaining a second blood sample from said patient and contacting said second blood sample with said compound, wherein said second blood sample is obtained at least 12 hours after obtaining said first blood sample;
iv) measuring the viability of leukocytes in said second blood sample; and
v) determining the efficacy of said therapy based on leukocyte viability, wherein an increase in said viability indicates that said therapy is efficacious.

49. The method of claim 48, wherein said viability is measured relative to the cell viability of leukocytes in a second blood sample obtained from a human not having or not predisposed to said autoimmune disease, wherein said second blood sample and said blood sample of step (a) are contacted with said compound in the same manner.

50. The method of claim 48, wherein said viability is a measure of autoimmune cell viability in said sample relative to a second blood element in said sample.

51. The method of claim 50, wherein said second blood element is a non-autoimmune leukocyte.

52. The method of claim 50, wherein said second blood element is an erythrocyte.

53. The method of claim 48, wherein said compound is administered to said patient.

54. The method of claim 48, wherein said autoimmune disease is Alopecia, Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barre, Hashimoto's Thyroiditis, Hypothyroidism, Idiopathic Pulmonary Fibrosis, Idiopathic Thrombocytopenia Purpura (ITP), IgA Nephropathy, insulin dependent Diabetes, Juvenile Arthritis, Lichen Planus, Lupus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjögren's Syndrome, Stiffman Syndrome, Takayasu Arteritis, Temporal Arteritis/Giant Cell Arteritis, Ulcerative Colitis, Uveitis, Vasculitis, Vitiligo, Wegener's Granulomatosis, or myasthenia gravis.

55. The method of claim 54, wherein said autoimmune disease is Insulin-dependent Diabetes.
